(19) United States
(12) Patent Application Publication
(10) Pub. No.: US 2023/0228703 A1
(43) Pub. Date: Jul. 20, 2023

(54) GAS SENSOR

(71) Applicant: NGK Insulators, Ltd., Nagoya-shi (JP)

(72) Inventors: Yusuke WATANABE, Nagoya-shi (JP); Yasumasa FUJIOKA, Nagoya-shi (JP); Yohei GORO, Nagoya-shi (JP); Akihiro NAKAJIMA, Nagoya-shi (JP); Shotaro NIIZUMA, Nagoya-shi (JP)

(21) Appl. No.: 18/154,370

(22) Filed: Jan. 13, 2023

(30) Foreign Application Priority Data
Jan. 14, 2022 (JP) 2022-004225
Dec. 8, 2022 (JP) 2022-196271

(51) Int. Cl. G01N 27/409; G01N 27/41; G01N 27/406; G01N 33/00; G01N 27/30

(52) U.S. Cl. CPC G01N 27/409; G01N 27/41; G01N 27/4067; G01N 33/0037; G01N 27/30

(57) ABSTRACT

Provided is a gas sensor element capable of realizing both highly accurate concentration measurement in environments where the concentration of a specific gas in a measurement target gas is high and highly accurate concentration measurement in environments where the concentration is low. A gas sensor according to one aspect of the present invention adjusts a sensor element drive temperature such that the value of cell resistance of a main pump cell is a predetermined value. Further, in the gas sensor according to one aspect of the present invention, the slope of the cell resistance of the main pump cell is larger than the slope of cell resistance of a measurement pump cell.

### CROSS-REFERENCE TO RELATED APPLICATION

The present application claims priority from Japanese application JP 2022-004225, filed on Jan. 14, 2022, and JP 2022-196271, filed Dec. 8, 2022, the contents of which are hereby incorporated by reference into this application.

### TECHNICAL FIELD

The present invention relates to a gas sensor.

### BACKGROUND ART

Conventionally, some known gas sensors for detecting the concentration of a specific gas, such as oxygen or NOₓ, in a measurement target gas, such as exhaust gas from an automobile, have a sensor element with an internally embedded heater in order to activate an oxygen ion-conductive solid electrolyte that constitutes the sensor element. For example, JP 10-318979A discloses a gas sensor that has a sensor element with an internally embedded heater and that controls electric power of the heater such that the impedance of a measurement pump cell is constant.

JP 10-318979A is an example of related art.

### SUMMARY OF THE INVENTION

With the tightening of automobile emission regulations and the like, it is predicted that gas sensors will be required to be capable of not only highly accurate concentration measurement in environments where the concentration of a specific gas in the measurement target gas is high in addition to environments where the concentration of the specific gas is low. Studies from this perspective by the inventor of the present application lead to the invention finding that, with conventional gas sensors having a structure such as that described above, it is difficult to realize highly accurate concentration measurement in both environments where the concentration of the specific gas in the measurement target gas is high and highly accurate concentration measurement in environments where the concentration is low. This is described in detail below.

First, the inventor found that, in gas sensors that measure the concentration of the specific gas in the measurement target gas, generally, changes in the offset value significantly affect the measurement accuracy in environments where the concentration is low. That is, the concentration to be measured varies by several ppm depending on the change in the offset value. Therefore, even if the variation in the offset value is constant, the lower the concentration of the specific gas in the measurement target gas is, the more significantly a change in the offset value affects the measurement accuracy. For example, when the concentration of the specific gas in the measurement target gas is 500 ppm, an error caused by a change in the offset value remains at 1% even if the offset value changes by 5 ppm. In contrast, when the concentration of the specific gas in the measurement target gas is 50 ppm, the error caused by a change in the offset value is 10% if the offset value changes by 5 ppm, and the change in the offset value significantly affects the measurement accuracy.

Here, one possible cause of the change in the offset value is a change in the temperature of the sensor element (specifically, electrodes such as a measurement electrode) due to the set temperature of the heater, the temperature of the measurement target gas, or the like. For this reason, the inventor examined suppressing the change in the offset value by lowering the temperature of the sensor element, i.e., lowering the temperature of the heater.

As a result, the inventor found an issue where simply lowering the temperature of the sensor element (electrodes) may reduce the measurement accuracy. That is, lowering the temperature of the sensor element increases the resistance to the reaction of an adjustment pump cell, which is an electrochemical pump cell for adjusting the oxygen concentration. Therefore, a pump voltage to be applied to the adjustment pump cell needs to be increased. However, there is a concern that, if the pump voltage to be applied to the adjustment pump cell is increased, the sensitivity to the specific gas may decrease as a result of the specific gas in the measurement target gas decomposing in the adjustment pump cell. Particularly, the measurement accuracy may deteriorate when the concentration of the specific gas is high.

The present invention has been made in view of the foregoing situation in one aspect, and aims to provide a gas sensor element capable of realizing highly accurate concentration measurement in both environments where the concentration of the specific gas in the measurement target gas is high and environments where the concentration is low.

To solve the above-described problems, the present invention adopts the following configuration.

A gas sensor according to a first aspect includes: a sensor element formed by stacking a plurality of solid electrolyte layers having oxygen ion conductivity, the sensor element including: an internal cavity into which a measurement target gas is to be introduced; a measurement pump cell being an electrochemical pump cell including: a measurement electrode located in the internal cavity; an outer pump electrode located in a region different from the internal cavity; and a solid electrolyte layer, of the plurality of solid electrolyte layers, that is present between the measurement electrode and the outer pump electrode; an adjustment pump cell being an electrochemical pump cell including: an inner pump electrode facing the internal cavity; the outer pump electrode, or a third electrode in contact with a solid electrolyte layer, of the plurality of solid electrolyte layers, and exposed to an external space; and a solid electrolyte layer, of the plurality of solid electrolyte layers, that is present between the inner pump electrode and the outer pump electrode or the third electrode; and a heater unit embedded in the sensor element and configured to heat the sensor element to a specific temperature; a detection unit configured to detect a value of cell resistance of the adjustment pump cell; and an adjustment unit configured to adjust the specific temperature such that the value of the cell resistance of the adjustment pump cell detected by the detection unit is a predetermined value, wherein a slope of the cell resistance of the adjustment pump cell with respect to input power to the heater unit is larger than a slope of cell resistance of the measurement pump cell with respect to the input power to the heater unit.

In this configuration, the specific temperature is adjusted, i.e., controlled such that the value of the cell resistance of the adjustment pump cell detected by the detection unit is the predetermined value. Further, the slope of the cell resistance of the adjustment pump cell with respect to the input power to the heater unit is larger than the slope of the cell resistance of the measurement pump cell with respect to the input power to the heater unit. In other words, as for the slope of the cell resistance with respect to the input power to the heater unit, the slope of the adjustment pump cell is larger than the slope of the measurement pump cell in this configuration.

The following effects are achieved by controlling the specific temperature to set the value of the cell resistance of the adjustment pump cell to the predetermined value. That is, the value of the cell resistance of the adjustment pump cell being kept at the predetermined value does not increase the resistance to the reaction of the adjustment pump cell, and eliminates the need to increase the pump voltage to be applied to the adjustment pump cell. Accordingly, it is possible to avoid a situation where, as a result of increasing the pump voltage to be applied to the adjustment pump cell, the specific gas in the measurement target gas is decomposed in the adjustment pump cell, resulting in deterioration of the measurement accuracy for the concentration of the specific gas, particularly when the concentration of the specific gas is high.

As for the slope of the cell resistance with respect to the input power, the slope of the adjustment pump cell is larger than the slope of the measurement pump cell. Therefore, a control can be performed with small input power such that the value of the cell resistance of the adjustment pump cell is the predetermined value.

Furthermore, since the slope of the cell resistance of the adjustment pump cell with respect to the input power is large, the change in the specific temperature can be reduced when the input power (i.e., the specific temperature) is changed such that the value of the cell resistance of the adjustment pump cell is the predetermined value. The change in the offset value can be suppressed by reducing the change in the specific temperature, thus enabling highly accurate concentration measurement even when the concentration of the specific gas in the measurement target gas is low.

In addition, as for the slope of the cell resistance with respect to the input power, the slope of the measurement pump cell is smaller than the slope of the adjustment pump cell. Therefore, the amount of change in the cell resistance of the measurement pump cell is small even if the temperature (e.g., the temperature of the measurement pump cell) changes, and the change in the offset value can be suppressed.

As described above, the gas sensor according to the first aspect can avoid a situation where the measurement accuracy deteriorates when the concentration of the specific gas in the measurement target gas is high, and can also realize highly accurate concentration measurement when the concentration of the specific gas in the measurement target gas is low. In other words, the gas sensor according to the first aspect can realize highly accurate concentration measurement in both environments where the concentration of the specific gas in the measurement target gas is high and environments where the concentration is low. Note that the slope of the cell resistance with respect to the input power to the heater unit refers to, for example, the slope of the cell resistance with respect to the input power to the heater unit in the atmosphere.

A gas sensor according to a second aspect may be the gas sensor according to the first aspect, wherein the slope of the cell resistance of the adjustment pump cell with respect to the input power to the heater unit is 1.5 to 1000 times the slope of the cell resistance of the measurement pump cell with respect to the input power to the heater unit. In this configuration, the slope of the cell resistance of the adjustment pump cell with respect to the input power to the heater unit is 1.5 to 1000 times the slope of the cell resistance of the measurement pump cell with respect to the input power to the heater unit. To realize highly accurate concentration measurement under both high and low concentrations, it is desirable to make the slope of the cell resistance of the adjustment pump cell with respect to the input power larger than the slope of the cell resistance of the measurement pump cell with respect to the input power, as mentioned above. The inventor confirmed through experiments that it is desirable to make the slope of the cell resistance of the adjustment pump cell with respect to the input power 1.5 to 1000 times the slope of the cell resistance of the measurement pump cell with respect to the input power. Accordingly, the gas sensor according to the second aspect can realize highly accurate concentration measurement under both high and low concentrations as a result of the adjustment pump cell and the measurement pump cell satisfying the aforementioned relationship regarding the slope of the cell resistance with respect to the input power.

A gas sensor according to a third aspect may be the gas sensor according to the first or second aspect, wherein the measurement electrode has an area larger than an area of the inner pump electrode. In this configuration, the area of the measurement electrode is larger than the area of the inner pump electrode. To realize highly accurate concentration measurement under both high and low concentrations, it is desirable to make the slope of the cell resistance of the adjustment pump cell with respect to the input power larger than the slope of the cell resistance of the measurement pump cell with respect to the input power, as mentioned above. The inventor confirmed through experiments that, to make the slope of the cell resistance of the adjustment pump cell with respect to the input power larger than the slope of the cell resistance of the measurement pump cell with respect to the input power, it is effective to make the area of the measurement electrode larger than the area of the inner pump electrode. Accordingly, the gas sensor according to the third aspect can realize highly accurate concentration measurement under both high and low concentrations by making the area of the measurement electrode larger than the area of the inner pump electrode.

A gas sensor according to a fourth aspect may be the gas sensor according to the first or second aspect, wherein the measurement electrode is thicker than the inner pump electrode. In this configuration, the measurement electrode is thicker than the inner pump electrode. To realize highly accurate concentration measurement under both high and low concentrations, it is desirable to make the slope of the cell resistance of the adjustment pump cell with respect to the input power larger than the slope of the cell resistance of the measurement pump cell with respect to the input power, as mentioned above. The inventor confirmed through experiments that, to make the slope of the cell resistance of the adjustment pump cell with respect to the input power larger than the slope of the cell resistance of the measurement pump cell with respect to the input power, it is effective to make the measurement electrode thicker than the inner pump electrode. Accordingly, the gas sensor according to the fourth aspect can realize highly accurate concentration measurement under both high and low concentrations by making the measurement electrode thicker than the inner pump electrode.

A gas sensor according to a fifth aspect may be the gas sensor according to any one of the first to fourth aspects, wherein the measurement electrode has a porosity lower than a porosity of the inner pump electrode. In this configuration, the porosity of the measurement electrode is lower than the porosity of the inner pump electrode. To realize highly accurate concentration measurement under both high and low concentrations, it is desirable to make the slope of the cell resistance of the adjustment pump cell with respect to the input power larger than the slope of the cell resistance of the measurement pump cell with respect to the input power, as mentioned above. The inventor confirmed through experiments that, to make the slope of the cell resistance of the adjustment pump cell with respect to the input power larger than the slope of the cell resistance of the measurement pump cell with respect to the input power, it is effective to make the porosity of the measurement electrode lower than the porosity of the inner pump electrode. Accordingly, the gas sensor according to the fifth aspect can realize highly accurate concentration measurement under both high and low concentrations by making the porosity of the measurement electrode lower than the porosity of the inner pump electrode.

A gas sensor according to a sixth aspect may be the gas sensor according to any one of the first to fifth aspects, wherein the measurement electrode and the inner pump electrode are cermet electrodes made of zirconia and precious metal, and a ratio of precious metal to zirconia in the measurement electrode is higher than a ratio of precious metal to zirconia in the inner pump electrode. In this configuration, the measurement electrode and the inner pump electrode are cermet electrodes made of zirconia and precious metal, and the ratio of precious metal to zirconia in the measurement electrode is higher than that in the inner pump electrode. To realize highly accurate concentration measurement under both high and low concentrations, it is desirable to make the slope of the cell resistance of the adjustment pump cell with respect to the input power larger than the slope of the cell resistance of the measurement pump cell with respect to the input power, as mentioned above. The inventor confirmed through experiments that, to make the slope of the cell resistance of the adjustment pump cell with respect to the input power larger than the slope of the cell resistance of the measurement pump cell with respect to the input power, it is effective to make the ratio of precious metal to zirconia in the measurement electrode higher than the ratio in the inner pump electrode. Accordingly, the gas sensor according to the sixth aspect can realize highly accurate concentration measurement under both high and low concentrations by making the ratio of precious metal to zirconia in the measurement electrode higher than the ratio in the inner pump electrode.

A gas sensor according to a seventh aspect may be the gas sensor according to any one of the first to sixth aspects, wherein the measurement electrode has an Au content lower than an Au content in the inner pump electrode. In this configuration, the Au content in the measurement electrode is lower than the Au content in the inner pump electrode. For example, the Au content in the measurement electrode may be 0%, i.e., the measurement electrode need not contain Au. Even when the measurement electrode contains Au, the Au content in the measurement electrode is lower than the Au content in the inner pump electrode. To realize highly accurate concentration measurement under both high and low concentrations, it is desirable to make the slope of the cell resistance of the adjustment pump cell with respect to the input power larger than the slope of the cell resistance of the measurement pump cell with respect to the input power, as mentioned above. The inventor confirmed through experiments that, to make the slope of the cell resistance of the adjustment pump cell with respect to the input power larger than the slope of the cell resistance of the measurement pump cell with respect to the input power, it is effective to configure the measurement electrode as follows. That is, the inventor confirmed that it is effective to make the Au content in the measurement electrode lower than the Au content in the inner pump electrode. That is, the inventor confirmed that it is effective that the inner pump electrode contains Au, while the measurement electrode does not contain Au. Further, the inventor confirmed that it is effective, even if the measurement electrode contains Au, to make the Au content in the measurement electrode lower than the Au content in the inner pump electrode. Accordingly, the gas sensor according to the seventh aspect can realize highly accurate concentration measurement under both high and low concentrations by making the Au content in the measurement electrode lower than the Au content in the inner pump electrode.

A gas sensor according to an eighth aspect may be the gas sensor according to any one of the first to seventh aspects, wherein a distance between the measurement electrode and the outer pump electrode is smaller than a distance between the inner pump electrode and the outer pump electrode or the third electrode. In this configuration, the distance between the measurement electrode and the outer pump electrode is smaller than the distance between the inner pump electrode and the outer pump electrode or the third electrode. To realize highly accurate concentration measurement under both high and low concentrations, it is desirable to make the slope of the cell resistance of the adjustment pump cell with respect to the input power larger than the slope of the cell resistance of the measurement pump cell with respect to the input power, as mentioned above. The inventor confirmed through experiments that, to make the slope of the cell resistance of the adjustment pump cell with respect to the input power larger than the slope of the cell resistance of the measurement pump cell with respect to the input power, it is effective to make the distance between the measurement electrode and the outer pump electrode smaller than the distance between the inner pump electrode and the outer pump electrode or the third electrode. Accordingly, the gas sensor according to the eighth aspect can realize highly accurate concentration measurement under both high and low concentrations by making the distance between the measurement electrode and the outer pump electrode smaller than the distance between the inner pump electrode and the outer pump electrode or the third electrode.

Note that as another mode of the gas sensor according to each of the above aspects, one aspect of the present invention may be an information processing method that realizes all or some of the above configurations, or may be a program, or may be a recording medium with such a program stored therein that can be read by a computer or any other device or machine. Here, the “recording medium that can be read by a computer or the like” refers to a medium in which information such as a program is stored by electrical, magnetic, optical, mechanical, or chemical action. The information processing method that realizes all or some of the above configurations may be referred to as, for example, a gas sensor control method or the like, as per the content of computations included. Similarly, the program that realizes all or some of the above configurations may be referred to as, for example, a gas sensor control program or the like.

For example, a gas sensor control method according to a ninth aspect is a method for controlling a gas sensor including a sensor element formed by stacking a plurality of solid electrolyte layers having oxygen ion conductivity, the sensor element including: an internal cavity into which a measurement target gas is to be introduced; a measurement pump cell being an electrochemical pump cell including: a measurement electrode located in the internal cavity; an outer pump electrode located in a region different from the internal cavity; and a solid electrolyte layer, of the plurality of solid electrolyte layers, that is present between the measurement electrode and the outer pump electrode; an adjustment pump cell being an electrochemical pump cell including: an inner pump electrode facing the internal cavity; the outer pump electrode, or a third electrode in contact with a solid electrolyte layer, of the plurality of solid electrolyte layers, and exposed to an external space; and a solid electrolyte layer, of the plurality of solid electrolyte layers, that is present between the inner pump electrode and the outer pump electrode or the third electrode; and a heater unit embedded in the sensor element and configured to heat the sensor element to a specific temperature, the method being an information processing method for executing: a detection step of detecting a value of cell resistance of the adjustment pump cell; and an adjustment step of adjusting the specific temperature such that the value of the cell resistance of the adjustment pump cell detected in the detection step is a predetermined value, wherein a slope of the cell resistance of the adjustment pump cell with respect to input power to the heater unit is larger than a slope of cell resistance of the measurement pump cell with respect to the input power to the heater unit.

According to the present invention, a gas sensor element can be provided that is capable of realizing highly accurate concentration measurement in both environments where the concentration of the specific gas in the measurement target gas is high and environments where the concentration is low.

### BRIEF DESCRIPTION OF THE DRAWINGS

FIG. 1 schematically shows an example configuration of a gas sensor and includes a vertical cross-sectional view of a sensor element as viewed in the longitudinal direction thereof.

FIG. 2 shows an example of a functional configuration of a controller included in the sensor in FIG. 1.

FIG. 3 shows the slopes of cell resistance of a main pump cell and a measurement pump cell with respect to input power to a heater unit in the sensor in FIG. 1.

FIG. 4 shows examples of numerical values of the electrode area, thickness, porosity, ratio of precious metal to zirconia, and Au content, as well as the inter-electrode distance in the main pump cell and the measurement pump cell of the sensor of in FIG. 1.

FIG. 5 shows examples of numerical values of the electrode area, thickness, porosity, ratio of precious metal to zirconia, and Au content, as well as the inter-electrode distance in the main pump cell and the measurement pump cell of a sensor according to a variation.

FIG. 6 shows an organized summary of experiments implemented by the inventor.

### EMBODIMENTS OF THE INVENTION

An embodiment according to an aspect of the present invention (hereinafter also referred to as “this embodiment”) will be described with reference to the drawings. However, the following embodiment is merely an example of the present invention in all respects. It goes without saying that various improvements and variations can be made without departing from the scope of the invention. In other words, specific configurations suitable for an embodiment may be adopted as appropriate to carry out of the present invention.

A gas sensor according to this embodiment is a sensor that detects NOₓ using a sensor element having an internally embedded heater unit for heating the sensor element to a specific temperature, and measures the concentration of the detected NO_{R}. The sensor element includes an adjustment pump cell, which is an electrochemical pump cell, and a measurement pump cell, which is an electrochemical pump cell into which a measurement target gas is introduced after oxygen contained in the measurement target gas has been pumped in the adjustment pump cell. The gas sensor according to this embodiment detects a value of cell resistance of the adjustment pump cell and adjusts the specific temperature such that the detected value of the cell resistance is a predetermined value. The slope of the cell resistance of the adjustment pump cell with respect to the input power to the heater unit is larger than the slope of the cell resistance of the measurement pump cell with respect to the input power to the heater unit.

The gas sensor according to this embodiment controls the specific temperature and sets the value of the cell resistance of the adjustment pump cell to a predetermined value. Thus, the resistance to the reaction of the adjustment pump cell does not increase, and the pump voltage to be applied to the adjustment pump cell need not be increased either. Therefore, the gas sensor according to this embodiment can avoid a situation where, as a result of increasing the pump voltage to be applied to the adjustment pump cell, NOₓ in the measurement target gas is decomposed in the adjustment pump cell, resulting in deterioration of the measurement accuracy for the NOₓ concentration, particularly deterioration of the measurement accuracy when the NOₓ concentration is high. Furthermore, the slope of the cell resistance of the adjustment pump cell with respect to the input power is large (compared to the slope of the cell resistance of the measurement pump cell with respect to the input power). Therefore, a control can be performed with a small amount of input power such that the value of the cell resistance of the adjustment pump cell is a predetermined value. Moreover, since the slope of the cell resistance of the adjustment pump cell with respect to the input power is large, the change in the specific temperature can be reduced when the input power (i.e., the specific temperature) is changed such that the value of the cell resistance of the adjustment pump cell is a predetermined value. The change in the offset value can be suppressed by reducing the change in the specific temperature, thus enabling highly accurate concentration measurement even when the NOₓ concentration in the measurement target gas is low.

As described above, the gas sensor according to an aspect of the present invention can avoid a situation where the measurement accuracy deteriorates when the NOₓ concentration in the measurement target gas is high, and can also realize highly accurate concentration measurement when the NOₓ concentration in the measurement target gas is low. In other words, the gas sensor according to an aspect of the present invention can realize highly accurate concentration measurement in both environments where the NOₓ concentration in the measurement target gas is high and environments where the concentration is low. An example of the gas sensor having the above configuration will be described below.

### Configuration Example

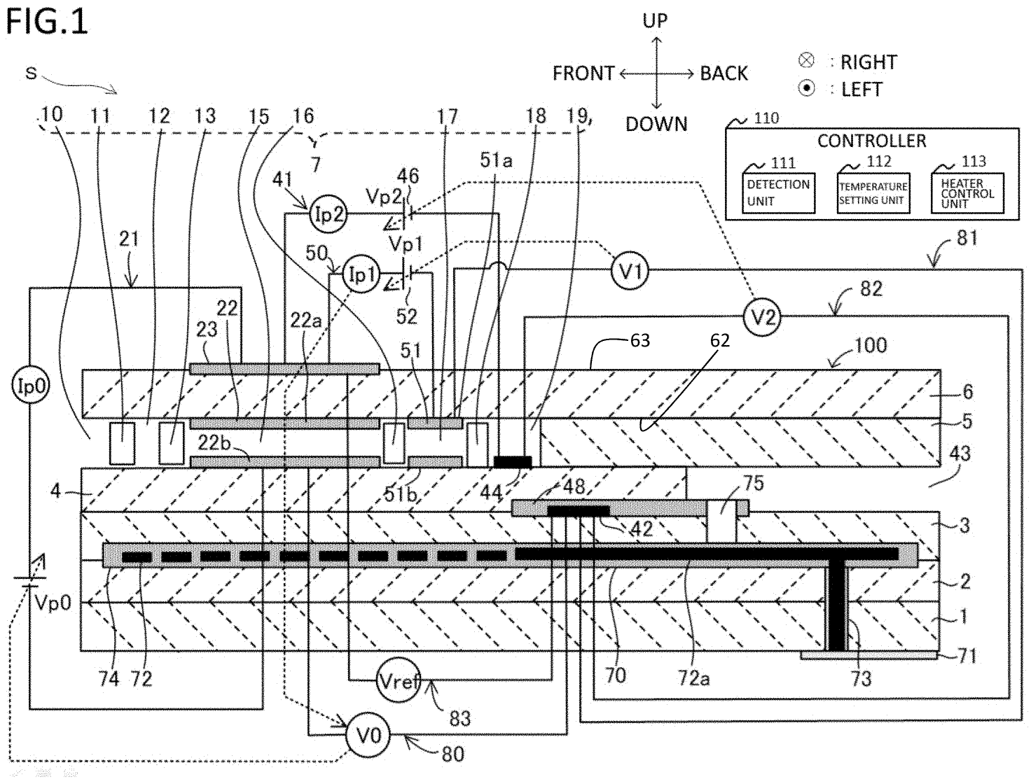

FIG. 1 schematically shows an example configuration of a gas sensor S and includes a vertical cross-sectional view of a gas sensor element 100 according to this embodiment as viewed in the longitudinal direction thereof. The gas sensor S includes the gas sensor element 100 and a controller 110, as shown in FIG. 1. The gas sensor element 100 has, for example, an elongated narrow plate body shape extending in the longitudinal direction (axial direction), and has, for example, a rectangular parallelepiped shape. The gas sensor element 100 illustrated in FIG. 1 has end portions in the longitudinal direction that are a leading end portion and a rear end portion. In the following description, the leading end portion corresponds to the left end portion in FIG. 1 (i.e., the end portion on the front side), and the rear end portion corresponds to the right end portion in FIG. 1 (i.e., the end portion on the rear side). However, the shape of the gas sensor element 100 need not be limited to this example, and may be selected as appropriate, as per the mode of implementation. Note that in the following description, the distal side of the paper plane of FIG. 1 corresponds to the right side of the gas sensor element 100, and the proximal side of the paper plane corresponds to the left side of the gas sensor element 100. The controller 110 has, as its functional elements, a detection unit 111, a temperature setting unit 112, and a heater control unit 113. The details of the gas sensor element 100 and the controller 110 will be described below.

### Gas Sensor Element

As illustrated in FIG. 1, the gas sensor element 100 includes a laminate constituted by a first substrate layer 1, a second substrate layer 2, a third substrate layer 3, a first solid electrolyte layer 4, a spacer layer 5, and a second solid electrolyte layer 6 that are stacked in this order from the lower side. These layers 1 to 6 are oxygen ion-conductive solid electrolyte layers made of zirconia (ZrO₂) or the like. The solid electrolyte that forms the layers 1 to 6 may be dense. Here, being dense means having a porosity of 5% or less.

The gas sensor element 100 is produced by, for example, performing steps such as predetermined processing and printing a wiring pattern on ceramic green sheets corresponding to respective layers, then stacking these layers, and firing and integrating them. As an example, the gas sensor element 100 is a laminate of a plurality of ceramic layers. In this embodiment, the upper face of the second solid electrolyte layer 6 constitutes the upper face of the gas sensor element 100, the lower face of the first substrate layer 1 constitutes the lower face of the gas sensor element 100, and side faces of the layers 1 to 6 constitute side faces of the gas sensor element 100.

In this embodiment, an internal space for receiving a measurement target gas from an external space is provided in one leading end portion of the gas sensor element 100 between a lower face 62 of the second solid electrolyte layer 6 and the upper face of the first solid electrolyte layer 4. The internal space according to this embodiment is formed such that a gas inlet 10, a first diffusion control portion 11, a buffer space 12, a second diffusion control portion 13, a first internal cavity 15, a third diffusion control portion 16, a second internal cavity 17, a fourth diffusion control portion 18, and a third internal cavity 19 are adjacent to each other in this order in a connected manner. In other words, the internal space according to this embodiment has a three-cavity structure (the first internal cavity 15, the second internal cavity 17, and the three internal cavity 19).

In one example, this internal space is formed by hollowing out a portion of the spacer layer 5. The upper portion of the internal space is demarcated by the lower face 62 of the second solid electrolyte layer 6. The lower portion of the internal space is demarcated by the upper face of the first solid electrolyte layer 4. The side portions of the internal space are demarcated by the side faces of the spacer layer 5.

The first diffusion control portion 11 is provided as two laterally elongated slits (the long sides of openings thereof extend in a direction perpendicular to the plane of FIG. 1). The second diffusion control portion 13, the third diffusion control portion 16, and the fourth diffusion control portion 18 are provided as holes whose length in a direction perpendicular to the plane of FIG. 1 are shorter than the first internal cavity 15, the second internal cavity 17, and the third internal cavity 19, respectively.

As illustrated in FIG. 1, the second diffusion control portion 13 and the third diffusion control portion 16 each may alternatively be provided as two laterally elongated slits (the long sides of openings thereof extend in a direction perpendicular to the plane of FIG. 1), similarly to the first diffusion control portion 11. On the other hand, the fourth diffusion control portion 18 may be provided as one laterally elongated slit (the long side of an opening thereof extends in a direction perpendicular to the plane of FIG. 1) that is formed as a gap below the lower face of the second solid electrolyte layer 6. That is, the fourth diffusion control portion 18 may be in contact with the upper face of the first solid electrolyte layer 4. The second diffusion control portion 13, the third diffusion control portion 16, and the fourth diffusion control portion 18 will be described later in detail. A region (internal space) from the gas inlet 10 to the third internal cavity 19 is referred to as a measurement target gas flow section 7.

A reference gas inlet space 43 having side portions demarcated by the side face of the first solid electrolyte layer 4 is provided between the upper face of the third substrate layer 3 and the lower face of the spacer layer 5, at a position farther from the leading end side (front side of the gas sensor element 100) than the measurement target gas flow section 7. A reference gas, such as the atmosphere, is introduced into the reference gas inlet space 43. However, the configuration of the gas sensor element 100 need not be limited to this example. As another example, the first solid electrolyte layer 4 may extend to the rear end of the gas sensor element 100, and the reference gas inlet space 43 may be omitted. In this case, an atmosphere inlet layer 48 may extend to the rear end of the gas sensor element 100.

The atmosphere inlet layer 48 is provided on a portion of the upper face of the third substrate layer 3 that is adjacent to the reference gas inlet space 43. The atmosphere inlet layer 48 is made of porous alumina, and the reference gas is introduced into the atmosphere inlet layer 48 via the reference gas inlet space 43. In addition, the atmosphere inlet layer 48 covers a reference electrode 42.

The reference electrode 42 is held between the first solid electrolyte layer 4 and the upper face of the third substrate layer 3, and is surrounded by the atmosphere inlet layer 48, which is connected to the reference gas inlet space 43. The reference electrode 42 is used to measure the oxygen concentration (oxygen partial pressure) in the first internal cavity 15 and the second internal cavity 17. The details will be described later.

The gas inlet 10 is a region of the measurement target gas flow section 7 that is open to the external space. The gas sensor element 100 is configured such that the measurement target gas is introduced from the external space to the inside through the gas inlet 10. In this embodiment, the gas inlet 10 is located in a leading end face (front face) of the gas sensor element 100, as illustrated in FIG. 1. In other words, the measurement target gas flow section 7 has an opening in the leading end face of the gas sensor element 100. However, it is not essential for the measurement target gas flow section 7 to have an opening in the leading end face of the gas sensor element 100, i.e., to dispose the gas inlet 10 in the leading end face of the gas sensor element 100 is not essential. The gas sensor element 100 need only be configured such that the measurement target gas can be introduced into the measurement target gas flow section 7 from the external space. For example, the gas inlet 10 may alternatively be located in the right face or left face of the gas sensor element 100.

The first diffusion control portion 11 is a region where predetermined diffusion resistance is applied to the measurement target gas introduced from the gas inlet 10.

The buffer space 12 is a space for guiding the measurement target gas introduced from the first diffusion control portion 11 to the second diffusion control portion 13.

The second diffusion control portion 13 is a region where predetermined diffusion resistance is applied to the measurement target gas introduced from the buffer space 12 to the first internal cavity 15.

When the measurement target gas is introduced into the first internal cavity 15 from the external space of the gas sensor element 100, there are cases where the measurement target gas is rapidly introduced into the gas sensor element 100 from the gas inlet 10 due to a change in the pressure of the measurement target gas in the external space (a pulsation of exhaust pressure in the case where the measurement target gas is exhaust gas from an automobile). Even in such cases, this configuration allows the introduced measurement target gas not to be introduced directly to the first internal cavity 15 but introduced to the first internal cavity 15 after a change in the concentration of the measurement target gas has been canceled out through the first diffusion control portion 11, the buffer space 12, and the second diffusion control portion 13. As a result, the change in the concentration of the measurement target gas introduced into the first internal cavity 15 is almost negligible.

The first internal cavity 15 is provided as a space for adjusting the oxygen partial pressure in the measurement target gas introduced through the second diffusion control portion 13. The oxygen partial pressure is adjusted by operation of the main pump cell 21.

The main pump cell 21 is an electrochemical pump cell that includes an inner pump electrode 22, an outer pump electrode 23, and the second solid electrolyte layer 6 held between these electrodes. The inner pump electrode 22 has a ceiling electrode portion 22a that is provided over the substantially entire surface of the lower face 62 of the second solid electrolyte layer 6 adjacent to (facing) the first internal cavity 15. The outer pump electrode 23 is provided in a region on an upper face 63 of the second solid electrolyte layer 6 that corresponds to the ceiling electrode portion 22a, and adjoins the external space. The main pump cell 21 is an example of an “adjustment pump cell”.

The inner pump electrode 22 is formed across the upper and lower solid electrolyte layers (the second solid electrolyte layer 6 and the first solid electrolyte layer 4) that demarcate the first internal cavity 15, and the spacer layer 5 that forms side walls of the first internal cavity 15. Specifically, the ceiling electrode portion 22a is formed on the lower face 62 of the second solid electrolyte layer 6 that forms a ceiling face of the first internal cavity 15, and a bottom electrode portion 22b is formed on the upper face of the first solid electrolyte layer 4 that forms a bottom face of the first internal cavity 15. Further, side electrode portions (not shown) that connect the ceiling electrode portion 22a to the bottom electrode portion 22b are formed on side wall faces (inner faces) of the spacer layer 5 that forms the two side wall portions of the first internal cavity 15. In other words, the inner pump electrode 22 is disposed in the form of a tunnel in the region where the side electrode portions are disposed. The inner pump electrode 22 is an example of an “inner pump electrode facing the internal cavity (measurement target gas flow section 7)”.

The inner pump electrode 22 and the outer pump electrode 23 are formed as porous cermet electrodes (e.g., cermet electrodes made of ZrO₂ and Pt containing 1% of Au). Note that the inner pump electrode 22, which comes into contact with the measurement target gas, is made of a material with a weakened capacity of reducing a nitrogen oxide (NOₓ) component in the measurement target gas.

The gas sensor element 100 is configured such that the main pump cell 21 applies a desired pump voltage Vp0 between the inner pump electrode 22 and the outer pump electrode 23 to cause a pump current Ip0 to flow in a positive or negative direction between the inner pump electrode 22 and the outer pump electrode 23, thereby enabling oxygen in the first internal cavity 15 to be pumped out to the external space, or oxygen in the external space to be pumped into the first internal cavity 15.

Furthermore, to detect the oxygen concentration (oxygen partial pressure) in the atmosphere in the first internal cavity 15, the inner pump electrode 22, the second solid electrolyte layer 6, the spacer layer 5, the first solid electrolyte layer 4, the third substrate layer 3, and the reference electrode 42 constitute an oxygen partial pressure detection sensor cell 80 for main pump control (i.e., an electrochemical sensor cell).

The gas sensor element 100 is capable of identifying the oxygen concentration (oxygen partial pressure) in the first internal cavity 15 by measuring an electromotive force V0 in the oxygen partial pressure detection sensor cell 80 for main pump control. Furthermore, the pump current Ip0 is controlled by performing feedback control on Vp0 so as to keep the electromotive force V0 constant. Accordingly, the oxygen concentration in the first internal cavity 15 can be kept at a predetermined constant value.

The third diffusion control portion 16 is a region where predetermined diffusion resistance is applied to the measurement target gas whose oxygen concentration (oxygen partial pressure) has been controlled in the first internal cavity 15 through the operation of the main pump cell 21, and the measurement target gas is guided to the second internal cavity 17.

The second internal cavity 17 is provided as a space for further adjusting the oxygen partial pressure in the measurement target gas introduced through the third diffusion control portion 16. The oxygen partial pressure is adjusted by operation of an auxiliary pump cell 50.

The auxiliary pump cell 50 is an auxiliary electrochemical pump cell constituted by an auxiliary pump electrode 51, the outer pump electrode 23 (not limited to the outer pump electrode 23 but need only be an appropriate electrode on the outer side of the gas sensor element 100), and the second solid electrolyte layer 6. The auxiliary pump electrode 51 has a ceiling electrode portion 51a located over a substantially entire portion facing the second internal cavity 17, of the lower face of the second solid electrolyte layer 6. The auxiliary pump cell 50 is an example of the “adjustment pump cell”. Also, the aforementioned “appropriate electrode on the outer side of the gas sensor element 100” is an example of a “third electrode in contact with the solid electrolyte layer and exposed to an external space”.

This auxiliary pump electrode 51 is disposed within the second internal cavity 17 in the form of a tunnel similar to the aforementioned inner pump electrode 22 provided within the first internal cavity 15. That is, the ceiling electrode portion 51a is formed on the lower face 62 of the second solid electrolyte layer 6 that forms the ceiling face of the second internal cavity 17, and a bottom electrode portion 51b is formed on the upper face of the first solid electrolyte layer 4 that forms the bottom face of the second internal cavity 17. Side electrode portions (not shown) that connect the ceiling electrode portion 51a to the bottom electrode portion 51b are formed on respective wall faces of the spacer layer 5 that form side walls of the second internal cavity 17. Thus, the auxiliary pump electrode 51 has a structure in the form of a tunnel. The auxiliary pump electrode 51 is an example of an “inner pump electrode facing an internal cavity (measurement target gas flow section 7)”.

Note that the auxiliary pump electrode 51 is also made of a material that has a weakened capacity of reducing a nitrogen oxide component in the measurement target gas, similarly to the internal pump electrode 22.

The gas sensor element 100 is configured such that the auxiliary pump cell 50 applies a desired voltage Vp1 between the auxiliary pump electrode 51 and the outer pump electrode 23, thereby enabling oxygen in the atmosphere in the second internal cavity 17 to be pumped out to the external space, or to be pumped into the second internal cavity 17 from the external space.

Furthermore, to control the oxygen partial pressure in the atmosphere in the second internal cavity 17, the auxiliary pump electrode 51, the reference electrode 42, the second solid electrolyte layer 6, the spacer layer 5, the first solid electrolyte layer 4, and the third substrate layer 3 constitute an oxygen partial pressure detection sensor cell 81 for auxiliary pump control (i.e., an electrochemical sensor cell).

Note that the auxiliary pump cell 50 performs pumping using a variable power source 52 whose voltage is controlled based on an electromotive force V1 detected by the oxygen partial pressure detection sensor cell 81 for auxiliary pump control. This controls the oxygen partial pressure in the atmosphere in the second internal cavity 17 to a partial pressure that is low enough to have substantially no impact on the NOₓ measurement.

Further, a pump current Ip1 is used together to control the electromotive force of the oxygen partial pressure detection sensor cell 80 for main pump control. Specifically, the pump current Ip1 is input as a control signal to the oxygen partial pressure detection sensor cell 80 for main pump control, and the electromotive force V0 is controlled so as to keep a constant gradient of the oxygen partial pressure in the measurement target gas introduced into the second internal cavity 17 from the third diffusion control portion 16. When this gas sensor is used as an NOₓ sensor, the oxygen concentration in the second internal cavity 17 is kept at a constant value of about 0.001 ppm by the operation of the main pump cell 21 and the auxiliary pump cell 50.

The fourth diffusion control portion 18 is a region where predetermined diffusion resistance is applied to the measurement target gas whose oxygen concentration (oxygen partial pressure) has been controlled by the operation of the auxiliary pump cell 50 in the second internal cavity 17, and this measurement target gas is guided to the third internal cavity 19.

The third internal cavity 19 is provided as a space for performing processing related to measurement of the nitrogen oxide (NOₓ) concentration in the measurement target gas introduced through the fourth diffusion control portion 18. The NOₓ concentration is measured by operation of a measurement pump cell 41. In this embodiment, the measurement target gas, which has been subjected to the pre-adjustment of the oxygen concentration (oxygen partial pressure) in the first internal cavity 15 and then introduced through the third diffusion control portion, is subjected to further adjustment of the oxygen partial pressure by the auxiliary pump cell 50 in the second internal cavity 17. This can accurately keep constant the oxygen concentration in the measurement target gas introduced into the third internal cavity 19 from the second internal cavity 17. Accordingly, the gas sensor element 100 according to this embodiment can accurately measure the NOₓ concentration.

The measurement pump cell 41 is used to measure the nitrogen oxide concentration in the measurement target gas in the third internal cavity 19. The measurement pump cell 41 is an electrochemical pump cell constituted by a measurement electrode 44, the outer pump electrode 23, the second solid electrolyte layer 6, the spacer layer 5, and the first solid electrolyte layer 4. In the example shown in FIG. 1, the measurement electrode 44 is located on the upper face of the first solid electrolyte layer 4 adjacent to (facing) the third internal cavity 19.

The measurement electrode 44 is a porous cermet electrode. The measurement electrode 44 also functions as an NOₓ reduction catalyst for reducing NOₓ that is present in the atmosphere in the third internal cavity 19. In the example shown in FIG. 1, the measurement electrode 44 is exposed in the third internal cavity 19. In another example, the measurement electrode 44 may be covered by a diffusion control portion. This diffusion control portion may be constituted by a porous film containing alumina (Al₂O₃) as a main component. The diffusion control portion serves to restrict the amount of NOₓ flowing into the measurement electrode 44, and also functions as a protective film for the measurement electrode 44.

The gas sensor element 100 is configured such that the measurement pump cell 41 can pump out oxygen generated through decomposition of nitrogen oxide in the atmosphere around the measurement electrode 44, and detect the amount of generated oxygen as a pump current Ip2.

To detect the oxygen partial pressure around the measurement electrode 44, the second solid electrolyte layer 6, the spacer layer 5, the first solid electrolyte layer 4, the third substrate layer 3, the measurement electrode 44, and the reference electrode 42 constitute an oxygen partial pressure detection sensor cell 82 for measurement pump control (i.e., an electrochemical sensor cell). A variable power source 46 is controlled based on a voltage (electromotive force) V2 detected by the oxygen partial pressure detection sensor cell 82 for measurement pump control.

The measurement target gas guided into the third internal cavity 19 reaches the measurement electrode 44 with the oxygen partial pressure controlled. Nitrogen oxide in the measurement target gas around the measurement electrode 44 is reduced to generate oxygen (2NO→N₂+O₂). The generated oxygen is pumped by the measurement pump cell 41. At that time, a voltage Vp2 of the variable power source is controlled such that the control voltage V2 detected by the oxygen partial pressure detection sensor cell 82 for measurement pump control is constant. The amount of oxygen generated around the measurement electrode 44 is proportional to the nitrogen oxide concentration in the measurement target gas. Therefore, the nitrogen oxide concentration in the measurement target gas is calculated using the pump current Ip2 in the measurement pump cell 41.

By combining the measurement electrode 44, the first solid electrolyte layer 4, the third substrate layer 3, and the reference electrode 42 to constitute an oxygen partial pressure detection means serving as an electrochemical sensor cell, it is possible to detect an electromotive force that corresponds to a difference between the amount of oxygen generated due to the reduction of an NOₓ component in the atmosphere around the measurement electrode 44 and the amount of oxygen contained in reference air. Thus, the concentration of the nitrogen oxide component in the measurement target gas can also be obtained.

Further, the second solid electrolyte layer 6, the spacer layer 5, the first solid electrolyte layer 4, the third substrate layer 3, the outer pump electrode 23, and the reference electrode 42 constitute an electrochemical sensor cell 83. The gas sensor element 100 is capable of detecting the oxygen partial pressure in the measurement target gas outside the sensor, based on an electromotive force Vref obtained by the sensor cell 83.

In the gas sensor element 100 having the above-described configuration, the measurement target gas whose oxygen partial pressure is always kept at a constant low value (a value that has no substantial impact on the NOₓ measurement) can be supplied to the measurement pump cell 41 by operating the main pump cell 21 and the auxiliary pump cell 50. Accordingly, the gas sensor element 100 is capable of identifying the nitrogen oxide concentration in the measurement target gas based on the pump current Ip2 that flows as a result of oxygen generated through the reduction of NOₓ being pumped out by the measurement pump cell 41, substantially in proportion to the nitrogen oxide concentration in the measurement target gas.

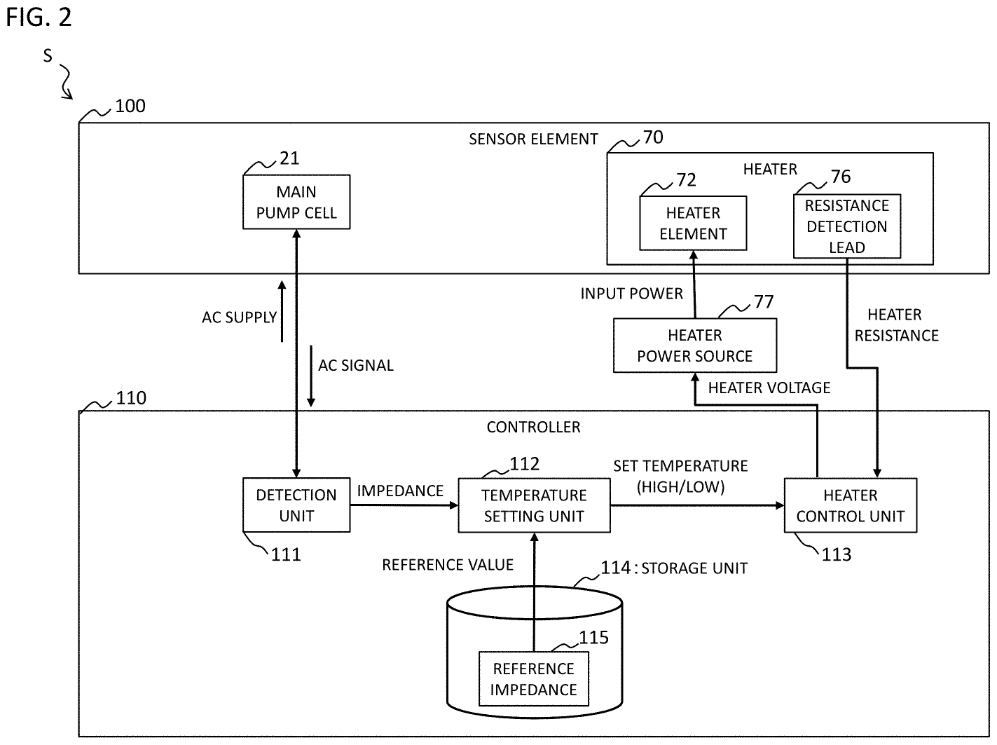

Furthermore, to improve the oxygen ion conductivity of the solid electrolyte, the gas sensor element 100 includes a heater 70 that serves to adjust the temperature by heating the gas sensor element 100 and retaining the temperature thereof. The heater 70 includes, as main components, heater electrodes 71 (for example, 71a, 71b, and 71c (not shown)), a heater element 72, heater leads 72a (for example, 72a1 and 72a2 (not shown)), through-holes 73, a heater insulating layer 74, and a heater resistance detection lead 76 (FIG. 2), which is not shown in FIG. 1. In the example in FIG. 1, the heater 70 also includes a pressure release hole 75.

The heater 70 is buried in a base portion of the gas sensor element 100, except for the heater electrodes 71. In this embodiment, the heater 70 is disposed at a position that is closer to the lower face of the gas sensor element 100 than to the upper face of the gas sensor element 100 in the thickness direction (vertical direction/stacking direction) of the gas sensor element 100. Note that the location of the heater 70 is not limited to this example, and may be selected as appropriate, as per the mode of implementation.

The heater electrodes 71 are electrodes formed in contact with the lower face of the first substrate layer 1 (the lower face of the gas sensor element 100). Electricity can be supplied from the outside to the heater 70 by connecting the heater electrodes 71 to an external power source.

The heater element 72 is an electrical resistor that is held from below and above by the second substrate layer 2 and the third substrate layer 3, i.e., heating resistors provided between the second substrate layer 2 and the third substrate layer 3. The heater element 72 is supplied electricity from a heater power source 77 (FIG. 2; not shown in FIG. 1) provided outside the gas sensor element 100 via an electricity flow path constituted by the heater electrodes 71, the through-holes 73, and the heater leads 72a, thereby generating heat to heat the solid electrolyte that forms the gas sensor element 100 and retain the temperature thereof. The heater element 72 is made of Pt, or contains Pt as a main component. The heater element 72 is buried in a predetermined area of the gas sensor element 100 on the side where the measurement target gas flow section 7 is located, and face the measurement target gas flow section 7 in the element thickness direction. Each heater element 72 has a thickness of about 10 μm to 20 μm, for example.

Two heater leads (for example, heater leads 72a1 and 72a2 (not shown)) connected to respective ends of each heater element 72 have substantially the same shape, i.e., the same resistance value. The heater leads 72a1 and 72a2 are connected respectively to different heater electrodes 71a and 71b (not shown) via corresponding through-holes 73.

Further, the heater resistance detection lead 76 is pulled out from a connecting portion between the heater element 72 and one of the heater leads, i.e., the heater lead 72a2. Note that the resistance value of the heater resistance detection lead 76 is negligible. The heater resistance detection lead 76 is connected to the heater electrode 71c (not shown) via a corresponding through-hole 73.

The heater element 72 is capable of adjusting the temperature of the entire gas sensor element 100 at a temperature that activates the solid electrolyte. That is, in the gas sensor element 100, each part of the gas sensor element 100 can be heated to a specific temperature and this temperature can be retained by causing a current to flow through the heater element 72 via the heater electrodes 71 to heat the heater element 72. Specifically, the gas sensor element 100 is heated such that the temperature of the solid electrolyte and the electrodes near the measurement target gas flow section 7 is about 700° C. to 900° C. (or 750° C. to 950° C.). This heating enhances the oxygen ion conductivity of the solid electrolyte constituting the base portion in the gas sensor element 100. Note that the heating temperature of the heater element 72 when the gas sensor S is used (i.e., when the gas sensor element 100 is driven) is referred to as a sensor element drive temperature in some cases.

The degree of heat generation by the heater element 72 is understood based on the magnitude of the resistance value (heater resistance) of the heater element 72. The heater resistance detection lead 76 is provided to measure the heater resistance.

The heater insulating layer 74 is an insulating layer formed so as to cover the heater element 72, e.g., an insulating layer that is formed on the upper and lower faces of the heater element 72 and made of an insulator such as alumina. The heater insulating layer 74 is formed for the purpose of achieving electrical insulation properties between the second substrate layer 2 and the heater element 72 and electrical insulation properties between the third substrate layer 3 and the heater element 72. The heater insulating layer 74 has a thickness of about 70 μm to 110 μm and is located at a position separated from the leading end face and side faces of the gas sensor element 100 by about 200 μm to 700 μm. Note that the thickness of the heater insulating layer 74 need not be constant, and may be different between a location where the heater element 72 is present and a location where the heater element 72 is not present.

The pressure release hole 75 is a region that passes through the third substrate layer 3 and is in communication with the reference gas inlet space 43. The pressure release hole 75 is formed for the purpose of mitigating the increase in the internal pressure due to a temperature rise in the heater insulating layer 74. Note that the provision of the pressure release hole 75 is not essential, and the pressure release hole 75 need not be provided.

### Controller

Next, the functions of the controller 110 will be described in detail. The controller 110 controls operation of each part of the gas sensor S, identifies the NOₓ concentration based on the pump current Ip2 flowing through the gas sensor element 100, and heats the gas sensor element 100 to a “specific temperature (sensor element drive temperature)” using the heater 70. The controller 110 is realized by a general-purpose or dedicated computer, and includes the detection unit 111, the temperature setting unit 112 (adjustment unit), and the heater control unit 113 as functional constituent elements realized by a CPU, memory, and the like of the computer, as illustrated in FIG. 1. Note that if the gas sensor S is for detecting and measuring NOₓ contained in exhaust gas from an automobile engine, and the gas sensor element 100 is attached to an exhaust path, some or all of the functions of the controller 110 may be realized by ECUs (electronic control units) installed in the automobile.

The functional blocks of the controller 110 in FIG. 1 and other diagrams include the detection unit 111, the temperature setting unit 112, and the heater control unit 113. However, the controller 110 may also include any functional block other than these functional blocks. For example, the controller 110 may include functional blocks for NOₓ detection, concentration calculation, or any other purposes. Specifically, the controller 110 may also include a functional block for controlling operation of each pump cell, a functional block for calculating NOₓ concentration, a functional block for comprehensively controlling operation of each part of the controller 110, or the like.

The detection unit 111 measures (detects) a value of the cell resistance (impedance) of the main pump cell 21, and notifies the temperature setting unit 112 of the measured value of the cell resistance of the main pump cell 21. For example, the detection unit 111 may supply an alternating current between the inner pump electrode 22 and the outer pump electrode 23 of the main pump cell 21, and convert an alternating current signal generated therebetween into a voltage signal at a level corresponding to the impedance therebetween.

Specifically, the detection unit 111 may be an impedance detection circuit that is inserted and connected between the inner pump electrode 22 and the outer pump electrode 23 of the main pump cell 21 and detects the impedance between the inner pump electrode 22 and the outer pump electrode 23. The detection unit 111 may also include an alternating-current generation circuit for supplying an alternating current between the inner pump electrode 22 and the outer pump electrode 23, and a signal detection circuit for detecting a voltage signal at a level corresponding to the impedance generated therebetween due to the supply of the alternating current therebetween. The signal detection circuit of the detection unit 111 may be constituted by a filter circuit (e.g., a low-pass filter, a band pass filter, etc.) that converts an alternating current signal generated between the inner pump electrode 22 and the outer pump electrode 23 into a voltage signal at a level corresponding to the impedance between the inner pump electrode 22 and the outer pump electrode 23. Note that the detection unit 111 may detect the cell resistance as follows. That is, for example, the detection unit 111 may obtain the cell resistance by measuring an I-V curve in the atmosphere and calculating the slope from the current value when the voltage is swept at 5 mV/s between 0 and 50 mV.

The temperature setting unit 112 (adjustment unit) sets the specific temperature (sensor element drive temperature) that the gas sensor element 100 reaches as a result of being heated by the heater 70. In particular, the temperature setting unit 112 sets the sensor element drive temperature based on the value of the cell resistance of the main pump cell 21 detected by the detection unit 111. Specifically, the temperature setting unit 112, when notified of the value of the cell resistance of the main pump cell 21 by the detection unit 111, references a storage unit 114 and acquires a reference impedance 115. The temperature setting unit 112 then calculates a difference between the acquired reference impedance 115 and the value of the cell resistance of the main pump cell 21 that the temperature setting unit 112 has been notified by the detection unit 111. The reference impedance 115 is a value preset as the value of the cell resistance that the main pump cell 21 is to exhibit in the gas sensor S, for example. The temperature setting unit 112 sets the sensor element drive temperature based on the calculated difference between the reference impedance 115 and the value of the cell resistance of the main pump cell 21. Specifically, the temperature setting unit 112 sets the sensor element drive temperature so as to reduce the difference between the reference impedance 115 and the value of the cell resistance of the main pump cell 21. In other words, the temperature setting unit 112 sets the sensor element drive temperature such that the reference impedance 115 is equal to the value of the cell resistance of the main pump cell 21.

For example, if the value of the cell resistance of the main pump cell 21 is smaller than the reference impedance 115, the temperature setting unit 112 lowers the sensor element drive temperature that has been set to that point in time so as to increase the value of the cell resistance of the main pump cell 21 and make it equal to the reference impedance 115. The temperature setting unit 112 notifies the heater control unit 113 of the new sensor element drive temperature that has been lowered such that the value of the cell resistance of the main pump cell 21 is equal to the reference impedance 115.

If, for example, the value of the cell resistance of the main pump cell 21 is larger than the reference impedance 115, the temperature setting unit 112 raises the sensor element drive temperature that has been set to that point in time so as to reduce the value of the cell resistance of the main pump cell 21 and make it equal to the reference impedance 115. The temperature setting unit 112 notifies the heater control unit 113 of the new sensor element drive temperature that has been raised such that the value of the cell resistance of the main pump cell 21 is equal to the reference impedance 115.

The heater control unit 113 controls the operation of the heater 70 based on the sensor element drive temperature that the heater control unit 113 has been notified by the temperature setting unit 112. For example, the heater control unit 113 controls a heater voltage applied to the heater power source 77 such that the value of heater resistance (the resistance of the heater element 72), which is obtained as a resistance value between the heater resistance detection lead 76 and the heater lead 72a, is a value corresponding to the specific temperature set by the temperature setting unit 112. The heater control unit 113 thus controls the power supply to the heater 70, i.e., controls the input power to the heater 70. The heater element 72 generates an amount of heat corresponding to the heater resistance that is controlled in the above-described manner. When the heater control unit 113 controls the heater resistance value in accordance with the specific temperature set by the temperature setting unit 112, the gas sensor element 100 is heated by the heater 70 and reaches the specific temperature set by the temperature setting unit 112. The input power to the heater 70 refers to the product of the voltage applied to the heater 70 (i.e., the voltage applied across the heater) and the current flowing through the heater 70 (i.e., the current flowing in the heater).

### Constant-Impedance Control for Auxiliary Pump Cell

FIG. 2 is a diagram illustrating an overview of constant-impedance control performed in the gas sensor S. Constant-impedance control refers to control (processing) for keeping the cell resistance (impedance) of the auxiliary pump cell (e.g., the main pump cell 21 in the example shown in FIG. 2) constant. In the constant-impedance control, first, the detection unit 111 measures (detects) the value of the cell resistance (impedance) of the main pump cell 21, and, for example, detects the impedance between the inner pump electrode 22 and the outer pump electrode 23 (detection step), as illustrated in FIG. 2. The detection unit 111 notifies the temperature setting unit 112 of the detected value of the cell resistance of the main pump cell 21.

The temperature setting unit 112 sets the specific temperature, i.e., the sensor element drive temperature to which the gas sensor element 100 is to reach as a result of being heated by the heater 70, based on the value of the cell resistance of the main pump cell 21 that the temperature setting unit 112 has been notified by the detection unit 111. Specifically, the temperature setting unit 112 sets the sensor element drive temperature such that the reference impedance 115 is equal to the value of the cell resistance of the main pump cell 21 (adjustment step).

Note that, if the value of the cell resistance of the main pump cell 21 detected by the detection unit 111 is equal to the reference impedance 115, the temperature setting unit 112 may set the same sensor element drive temperature as the sensor element drive temperature that has been set to this point in time. The temperature setting unit 112 notifies the heater control unit 113 of the set sensor element drive temperature.

The heater control unit 113 controls the operation of the heater 70 based on the sensor element drive temperature that the heater control unit 113 has been notified by the temperature setting unit 112. For example, the heater control unit 113 controls the heater voltage to be applied to the heater power source 77 such that the value of heater resistance (the resistance of the heater element 72) is a value corresponding to the sensor element drive temperature that the heater control unit 113 has been notified by the temperature setting unit 112. The heater control unit 113 controls the input power (power supply) from the heater power source 77 to the heater 70, and the heater 70 heats the gas sensor element 100 such that the temperature of the gas sensor element 100 is the sensor element drive temperature set by the temperature setting unit 112.

Note that the above description is of an example where the main pump cell 21 is the adjustment pump cell whose value of the cell resistance (impedance) is detected by the detection unit 111. However, the auxiliary pump cell 50 may alternatively be the adjustment pump cell whose value of the cell resistance is detected by the detection unit 111. That is, the detection unit 111 may measure (detect) the value of the cell resistance of the auxiliary pump cell 50 as the value of the cell resistance (impedance) of the adjustment pump cell, and notify the temperature setting unit 112 of the measured value of the cell resistance of the auxiliary pump cell 50. For example, the detection unit 111 may supply an alternating current between the auxiliary pump electrode 51 and the outer pump electrode 23 (or the third electrode) of the auxiliary pump cell 50, and convert an alternating current signal generated therebetween into a voltage signal at a level corresponding to the impedance therebetween. Specifically, the detection unit 111 may be an impedance detection circuit that is inserted and connected between the auxiliary pump electrode 51 and the outer pump electrode 23 of the auxiliary pump cell 50 and detects the impedance between the auxiliary pump electrode 51 and the outer pump electrode 23. The method by which the detection unit 111 detects the value of the cell resistance of the auxiliary pump cell 50 is the same as the method by which the detection unit 111 detects the value of the cell resistance of the main pump cell 21, and therefore the details thereof are omitted.

Similarly, the auxiliary pump cell 50 may be the adjustment pump cell whose value of the cell resistance is made equal to the reference impedance 115 by the temperature setting unit 112 adjusting (controlling) the sensor element drive temperature. That is, the temperature setting unit 112 may set the sensor element drive temperature such that the reference impedance 115 is equal to the value of the cell resistance of the auxiliary pump cell 50. In this case, the reference impedance 115 is a value that is preset as the value of the cell resistance that the auxiliary pump cell 50 is to exhibit in the gas sensor S, for example.

Furthermore, the detection unit 111 may also measure (detect) the values of the cell resistance of the main pump cell 21 and the auxiliary pump cell 50 as the values of the cell resistance (impedance) of the adjustment pump cells. In this case, the detection unit 111 notifies the temperature setting unit 112 of the measured values of the cell resistance of the main pump cell 21 and the auxiliary pump cell 50. The temperature setting unit 112 may set the sensor element drive temperature such that the reference impedance 115 that is preset as the value of the cell resistance that the main pump cell 21 is to exhibit in the gas sensor S is equal to the value of the cell resistance of the main pump cell 21, and such that the reference impedance 115 that is preset as the value of the cell resistance that the auxiliary pump cell 50 is to exhibit in the gas sensor S is equal to the value of the cell resistance of the auxiliary pump cell 50.

That is, in the gas sensor S, the value of the cell resistance of at least either the main pump cell 21 or the auxiliary pump cell 50 is detected as the value of the cell resistance of the adjustment pump cell. Then, the sensor element drive temperature is set such that the detected value of the cell resistance of the adjustment pump cell is equal to the reference impedance 115 that is preset as the value of the cell resistance that the adjustment pump cell is to exhibit in the gas sensor S.

Slopes of Cell Resistance of Measurement Pump Cell and Adjustment Pump Cell with Respect to Input Power

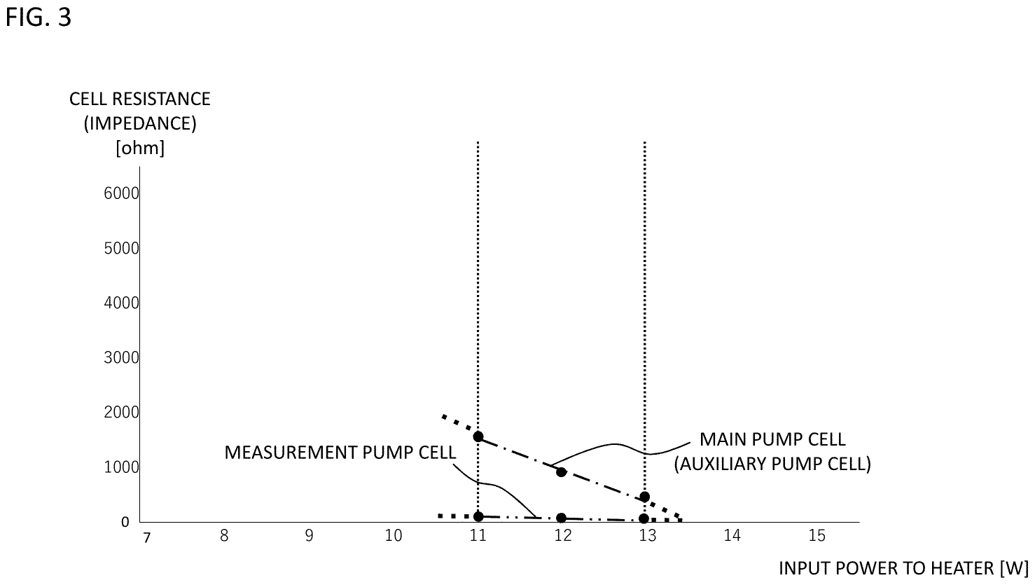

FIG. 3 shows a conceptual example of the slopes of the cell resistance of the measurement pump cell 41 and the main pump cell 21 with respect to the input power (power supply) to the heater 70 in the gas sensor S. Note that the slope of the cell resistance with respect to the input power to the heater 70 refers to, for example, the slope of the cell resistance with respect to the input power to the heater 70 in the atmosphere.

In the gas sensor S, the slope of the cell resistance (impedance) [ohm] of the measurement pump cell 41 with respect to the input power [W] to the heater 70 is about 10 [ohm/W] with the input power to the heater 70 in the range from 11.5 to 13.5, as illustrated in FIG. 3. That is, the impedance between the measurement electrode 44 and the outer pump electrode 23 with respect to the input power to the heater 70 is about 10 [ohm/W] with the input power in the range from 11.5 to 13.5.

In contrast, in the gas sensor S, the slope of the cell resistance (impedance) [ohm] of the main pump cell 21 with respect to the input power [W] to the heater 70 is about 600 [ohm/W] with the input power to the heater 70 in the range from 11.5 to 13.5. That is, the impedance between the inner pump electrode 22 and the outer pump electrode 23 with respect to the input power to the heater 70 is about 600 [ohm/W] with the input power in the range from 11.5 to 13.5.

Therefore, in the gas sensor S, the slope of the cell resistance of the main pump cell 21 with respect to the input power to the heater 70 is larger than the slope of the cell resistance of the measurement pump cell 41 with respect to the input power to the heater 70.

In the gas sensor S, the sensor element drive temperature is adjusted, i.e., controlled such that the value of the cell resistance of the main pump cell 21 (adjustment pump cell) detected by the detection unit 111 is the predetermined value (reference impedance 115), as mentioned above. Further, the slope of the cell resistance of the main pump cell 21 with respect to the input power to the heater 70 is larger than the slope of the cell resistance of the measurement pump cell 41 with respect to the input power to the heater 70. In other words, in this configuration, the slope of the cell resistance of the main pump cell 21 with respect to the input power to the heater 70 is larger than that of the measurement pump cell 41.

The following effects are achieved by controlling the sensor element drive temperature and setting the value of the cell resistance of the main pump cell 21 to the predetermined value. That is, the value of the cell resistance of the main pump cell 21 being kept at the predetermined value does not increase resistance to the reaction of the main pump cell 21, and eliminates the need to increase the pump voltage to be applied to the main pump cell 21. Accordingly, it is possible to avoid a situation where, as a result of increasing the pump voltage (pump voltage Vp0) to be applied to the main pump cell 21, NOₓ in the measurement target gas is decomposed in the main pump cell 21, resulting in deterioration of the measurement accuracy for the NOₓ concentration, particularly when the NOₓ concentration is high.

As for the slope of the cell resistance with respect to the input power to the heater 70, the slope of the main pump cell 21 is large (compared with the slope of the measurement pump cell 41). Therefore, a control can be performed with a small amount of input power such that the value of the cell resistance of the main pump cell 21 is the predetermined value.

Furthermore, since the slope of the cell resistance of the main pump cell 21 with respect to the input power to the heater 70 is large, the change in the sensor element drive temperature can be reduced when the input power (i.e., the sensor element drive temperature) is changed such that the value of the cell resistance of the main pump cell 21 is the predetermined value. The change in the offset value can be suppressed by reducing the change in the sensor element drive temperature, and highly accurate concentration measurement can also be realized when the NOₓ concentration in the measurement target gas is low.

As described above, the gas sensor S can avoid a situation where the measurement accuracy deteriorates when the NOₓ concentration in the measurement target gas is high, and also realize highly accurate concentration measurement when the NOₓ concentration in the measurement target gas is low. In other words, the gas sensor S can realize both highly accurate concentration measurement in environments where the NOₓ concentration in the measurement target gas is high and highly accurate concentration measurement in environments where the concentration is low.

Note that the value of the cell resistance of the measurement pump cell 41 may alternatively be measured using the following impedance detection circuit, for example. That is, the value of the cell resistance of the measurement pump cell 41 may be measured using an impedance detection circuit that is inserted and connected between the measurement electrode 44 of the measurement pump cell 41 and the outer pump electrode 23 and detects the impedance between the measurement electrode 44 and the outer pump electrode 23. This impedance detection circuit may include an alternating-current generation circuit that supplies an alternating current between the measurement electrode 44 and the outer pump electrode 23, and a signal detection circuit that detects a voltage signal at a level corresponding to the impedance generated therebetween due to the alternating current supplied therebetween. This signal detection circuit can be constituted by a filter circuit (e.g., a low-pass filter, a band pass filter, etc.) that converts an alternating-current signal generated between the measurement electrode 44 and the outer pump electrode 23 to a voltage signal at a level corresponding to the impedance between the measurement electrode 44 and the outer pump electrode 23.

The above description is of an example where the main pump cell 21 is the adjustment pump cell whose slope of the cell resistance (impedance) with respect to the input power to the heater 70 is larger than that of the measurement pump cell 41. However, the auxiliary pump cell 50 may alternatively be the adjustment pump cell whose slope of the cell resistance (slope of the cell resistance with respect to the input power to the heater 70) is larger than that of the measurement pump cell 41. That is, the slope of the cell resistance of the auxiliary pump cell 50 with respect to the input power to the heater 70 may be larger than the slope of the cell resistance of the measurement pump cell 41 with respect to the input power to the heater 70.

As for the slope of the cell resistance (impedance) with respect to the input power to the heater 70, both the main pump cell 21 and the auxiliary pump cell 50 may have a larger slope than that of the measurement pump cell 41. That is, both the slope of the cell resistance of the main pump cell 21 with respect to the input power to the heater 70 and the slope of the cell resistance of the auxiliary pump cell 50 with respect to the input power to the heater 70 may be larger than the slope of the cell resistance of the measurement pump cell 41 with respect to the input power to the heater 70.

In the gas sensor S, the slope of the cell resistance (the slope of the cell resistance with respect to the input power to the heater 70) of at least either the main pump cell 21 or the auxiliary pump cell 50 need only be larger than the slope of the cell resistance of the measurement pump cell 41.

### Ratio Between Slope of Cell Resistance of the Measurement Pump Cell and Slope of Cell Resistance of Adjustment Pump Cell

In the gas sensor S, the slope of the cell resistance [ohm] of the measurement pump cell 41 with respect to the input power [W] to the heater 70 is about 10 [ohm/W] with the input power to the heater 70 in the range from 11.5 to 13.5, as mentioned above. The slope of the cell resistance [ohm] of the main pump cell 21 with respect to the input power [W] to the heater 70 is about 600 [ohm/W] with the input power to the heater 70 in the range from 11.5 to 13.5.

Accordingly, in the gas sensor S, the slope of the cell resistance of the main pump cell 21 with respect to the input power to the heater 70 is about 60 times the slope of the cell resistance of the measurement pump cell 41 with respect to the input power to the heater 70. In other words, in the gas sensor S, the slope of the cell resistance of the main pump cell 21 with respect to the input power to the heater 70 takes a value that is 1.5 to 1000 times the slope of the cell resistance of the measurement pump cell 41 with respect to the input power to the heater 70.

As mentioned above, to realize both highly accurate concentration measurement in environments where the NOₓ concentration in the measurement target gas is high and highly accurate concentration measurement in environments where the concentration is low, it is desirable to make the slope of the cell resistance of the adjustment pump cell (e.g., the main pump cell 21) with respect to the input power to the heater 70 larger than the slope of the cell resistance of the measurement pump cell 41 with respect to the input power. In particular, the inventor confirmed through experiments that it is desirable that the slope of the cell resistance of the adjustment pump cell with respect to the input power to the heater 70 is 1.5 to 1000 times the slope of the cell resistance of the measurement pump cell 41 with respect to the input power to the heater 70. For example, the inventor confirmed that it is desirable that the slope of the cell resistance of the main pump cell 21 with respect to the input power to the heater 70 is 1.5 to 1000 times the slope of the cell resistance of the measurement pump cell 41 with respect to the input power to the heater 70.

In the gas sensor S, the slope of the cell resistance of the main pump cell 21 with respect to the input power to the heater 70 is about 60 times the slope of the cell resistance of the measurement pump cell 41 with respect to the input power to the heater 70, as mentioned above. Accordingly, in the gas sensor S, the slope of the cell resistance of the main pump cell 21 takes a value that is 1.5 to 1000 times the slope of the cell resistance of the measurement pump cell 41 with respect to the input power to the heater 70, thus enabling highly accurate concentration measurement under both high and low concentrations.

Note that the above description is of an example where the main pump cell 21 is the adjustment pump cell whose slope of the cell resistance (the slope of the cell resistance with respect to the input power to the heater 70) takes a value that is 1.5 to 1000 times the slope of the cell resistance of the measurement pump cell 41. However, the auxiliary pump cell 50 may alternatively be the adjustment pump cell whose slope of the cell resistance takes a value that is 1.5 to 1000 times the slope of the cell resistance of the measurement pump cell 41. That is, the slope of the cell resistance of the auxiliary pump cell 50 with respect to the input power to the heater 70 may be 1.5 to 1000 times the slope of the cell resistance of the measurement pump cell 41 with respect to the input power to the heater 70.

Further, the slopes of the cell resistance (the slopes of the cell resistance with respect to the input power to the heater 70) of both the main pump cell 21 and the auxiliary pump cell 50 may be 1.5 to 1000 times the slope of the cell resistance of the measurement pump cell 41. That is, the value of the slope of the cell resistance of the main pump cell 21 with respect to the input power to the heater 70 and the value of the slope of the cell resistance of the auxiliary pump cell 50 with respect to the input power to the heater 70 both may be values that are 1.5 to 1000 times the slope of the cell resistance of the measurement pump cell 41 with respect to the input power to the heater 70.

In the gas sensor S, the slope of the cell resistance (the slope of the cell resistance with respect to the input power to the heater 70) of at least either the main pump cell 21 or the auxiliary pump cell 50 need only take a value that is 1.5 to 1000 times the slope of the cell resistance of the measurement pump cell 41.

### Parameters for Measurement Pump Cell and Adjustment Pump Cell

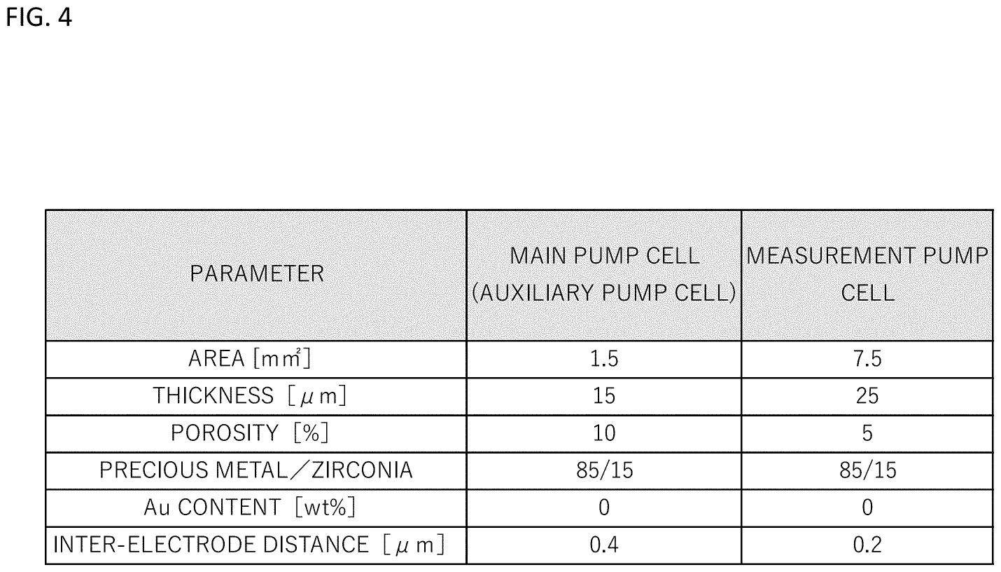

FIG. 4 shows examples of numerical values of the electrode area, thickness, porosity, ratio of precious metal to zirconia, and Au content, as well as the inter-electrode distance in the measurement pump cell 41 and the main pump cell 21 in the gas sensor S.

### Regarding Electrode Area

In the gas sensor S, the electrode area in the main pump cell 21, i.e., the area [mm²] of the inner pump electrode 22 (specifically, the area of the face thereof that is exposed to the measurement target gas) is 1.5, as illustrated in FIG. 4. Meanwhile, the electrode area in the measurement pump cell 41, i.e., the area [mm²] of the measurement electrode 44 (specifically, the area of the face thereof that is exposed to the measurement target gas) is 7.5. Therefore, in the gas sensor S, the area of the measurement electrode 44 (the area of the face thereof that is exposed to the measurement target gas) is larger than the area of the inner pump electrode 22 (the area of the face thereof that is exposed to the measurement target gas).

As mentioned above, to realize both highly accurate concentration measurement in environments where the NOₓ concentration in the measurement target gas is high and highly accurate concentration measurement in environments where the concentration is low, it is desirable to make the slope of the cell resistance of the adjustment pump cell (e.g., the main pump cell 21) with respect to the input power to the heater 70 larger than the slope of the cell resistance of the measurement pump cell 41 with respect to the input power. The inventor confirmed through experiments that it is effective to make the area of the measurement electrode 44 larger than the area of the inner pump electrode 22 in order to make the slope of the cell resistance of the main pump cell 21 with respect to the input power to the heater 70 larger than the slope of the cell resistance of the measurement pump cell 41 with respect to the input power. Accordingly, the area of the measurement electrode 44 being larger than the area of the inner pump electrode 22 enables the gas sensor S to realize highly accurate concentration measurement under both high and low concentrations.

### Regarding Electrode Thickness

In the gas sensor S, the electrode thickness in the main pump cell 21, i.e., the thickness [μm] of the inner pump electrode 22 is 15, as illustrated in FIG. 4. Meanwhile, the electrode thickness in the measurement pump cell 41, i.e., the thickness [μm] of the measurement electrode 44 is 25. Therefore, in the gas sensor S, the measurement electrode 44 is thicker than the inner pump electrode 22.

As mentioned above, to realize both highly accurate concentration measurement in environments where the NOₓ concentration in the measurement target gas is high and highly accurate concentration measurement in environments where the concentration is low, it is desirable to make the slope of the cell resistance of the adjustment pump cell (e.g., the main pump cell 21) with respect to the input power to the heater 70 larger than the slope of the cell resistance of the measurement pump cell 41 with respect to the input power. The inventor confirmed through experiments that it is effective to make the measurement electrode 44 thicker than the inner pump electrode 22 in order to make the slope of the cell resistance of the main pump cell 21 with respect to the input power to the heater 70 larger than the slope of the cell resistance of the measurement pump cell 41 with respect to the input power. Accordingly, the measurement electrode 44 being thicker than the inner pump electrode 22 enables the gas sensor S to realize highly accurate concentration measurement under both high and low concentrations.

### Regarding Electrode Porosity

In the gas sensor S, the electrode porosity in the main pump cell 21, i.e., the porosity [%] of the inner pump electrode 22 is 10, as illustrated in FIG. 4. Meanwhile, the electrode porosity in the measurement pump cell 41, i.e., the porosity [%] of the measurement electrode 44 is 5. Therefore, in the gas sensor S, the porosity of the measurement electrode 44 is lower than the porosity of the inner pump electrode 22.

As mentioned above, to realize both highly accurate concentration measurement in environments where the NOₓ concentration in the measurement target gas is high and highly accurate concentration measurement in environments where the concentration is low, it is desirable to make the slope of the cell resistance of the adjustment pump cell (e.g., the main pump cell 21) with respect to the input power to the heater 70 larger than the slope of the cell resistance of the measurement pump cell 41 with respect to the input power. The inventor confirmed through experiments that it is effective to make the porosity of the measurement electrode 44 lower than the porosity of the inner pump electrode 22 in order to make the slope of the cell resistance of the main pump cell 21 with respect to the input power to the heater 70 larger than the slope of the cell resistance of the measurement pump cell 41 with respect to the input power. Accordingly, the porosity of the measurement electrode 44 being lower than the porosity of the inner pump electrode 22 enables the gas sensor S to realize highly accurate concentration measurement under both high and low concentrations.

Note that the porosities of the inner pump electrode 22, the measurement electrode 44, the auxiliary pump electrode 51, and other electrodes in the gas sensor S are values measured by analyzing an SEM image obtained by observing the inner pump electrode 22, the measurement electrode 44, the auxiliary pump electrode 51, and other electrodes, using a SEM (scanning electron microscope).

### Regarding Ratio Between Precious Metal and Zirconia in Electrodes

In the gas sensor S, both the inner pump electrode 22 and the measurement electrode 44 are cermet electrodes made of zirconia and precious metal. In the gas sensor S, the ratio between precious metal and zirconia in the electrode of the main pump cell 21, i.e., the ratio between precious metal and zirconia in the inner pump electrode 22 is 85 (precious metal):15 (zirconia), as illustrated in FIG. 4. Meanwhile, the ratio between precious metal and zirconia in the electrode of the measurement pump cell 41, i.e., the ratio between precious metal and zirconia in the measurement electrode 44 is 85 (precious metal):15 (zirconia). Therefore, in the gas sensor S, the ratio of precious metal to zirconia in the measurement electrode 44 is the same as the ratio of precious metal to zirconia in the inner pump electrode 22.

As mentioned above, to realize both highly accurate concentration measurement in environments where the NOₓ concentration in the measurement target gas is high and highly accurate concentration measurement in environments where the concentration is low, it is desirable to make the slope of the cell resistance of the adjustment pump cell (e.g., the main pump cell 21) with respect to the input power to the heater 70 larger than the slope of the cell resistance of the measurement pump cell 41 with respect to the input power. The inventor confirmed through experiments that it is effective to make the ratio of precious metal to zirconia in the measurement electrode 44 higher than the ratio in the inner pump electrode 22 in order to make the slope of the cell resistance of the main pump cell 21 with respect to the input power to the heater 70 larger than the slope of the cell resistance of the measurement pump cell 41 with respect to the input power. Accordingly, the ratio of precious metal to zirconia in the measurement electrode 44 being higher than the ratio in the inner pump electrode 22 enables the gas sensor S to realize highly accurate concentration measurement under both high and low concentrations.

### Regarding Au Content in Electrodes

In the gas sensor S, the Au content in the electrode of the main pump cell 21, i.e., the Au content [wt %] in the inner pump electrode 22 is 0, as illustrated in FIG. 4. Meanwhile, the Au content in the electrode of the measurement pump cell 41, i.e., the Au content [wt %] in the measurement electrode 44 is 0. Therefore, in the gas sensor S, the Au content in the measurement electrode 44 is the same as the Au content in the inner pump electrode 22.

As mentioned above, to realize both highly accurate concentration measurement in environments where the NOₓ concentration in the measurement target gas is high and highly accurate concentration measurement in environments where the concentration is low, it is desirable to make the slope of the cell resistance of the adjustment pump cell (e.g., the main pump cell 21) with respect to the input power to the heater 70 larger than the slope of the cell resistance of the measurement pump cell 41 with respect to the input power. The inventor confirmed through experiments that it is effective to configure the measurement electrode 44 as follows in order to make the slope of the cell resistance of the main pump cell 21 with respect to the input power to the heater 70 larger than the slope of the cell resistance of the measurement pump cell 41 with respect to the input power. That is, the inventor confirmed that it is effective to make the Au content in the measurement electrode 44 lower than the Au content in the inner pump electrode 22. That is, the inventor confirmed that it is effective that the inner pump electrode 22 contains Au, while the measurement electrode 44 does not contain Au. Further, the inventor confirmed that it is effective, even if the measurement electrode 44 contains Au, to make the Au content in the measurement electrode 44 lower than the Au content in the inner pump electrode 22. Accordingly, the Au content in the measurement electrode 44 being lower than the Au content in the inner pump electrode 22 enables the gas sensor S to realize highly accurate concentration measurement under both high and low concentrations.

### Regarding Inter-Electrode Distance

In the gas sensor S, the inter-electrode distance in the main pump cell 21, i.e., the distance [μm] between the inner pump electrodes 22 and the outer pump electrode 23 is 0.4, as illustrated in FIG. 4. Meanwhile, the inter-electrode distance in the measurement pump cell 41, i.e., the distance [μm] between the measurement electrode 44 and the outer pump electrode 23 is 0.2. Therefore, in the gas sensor S, the inter-electrode distance in the measurement pump cell 41 (the distance between the measurement electrode 44 and the outer pump electrode 23) is smaller (shorter) than the inter-electrode distance in the main pump cell 21 (the distance between the inner pump electrode 22 and the outer pump electrode 23).

As mentioned above, to realize both highly accurate concentration measurement in environments where the NOₓ concentration in the measurement target gas is high and highly accurate concentration measurement in environments where the concentration is low, it is desirable to make the slope of the cell resistance of the adjustment pump cell (e.g., the main pump cell 21) with respect to the input power to the heater 70 larger than the slope of the cell resistance of the measurement pump cell 41 with respect to the input power. The inventor confirmed through experiments that it is effective to make the inter-electrode distance in the measurement pump cell 41 smaller (shorter) than the inter-electrode distance in the main pump cell 21 in order to make the slope of the cell resistance of the main pump cell 21 with respect to the input power to the heater 70 larger than the slope of the cell resistance of the measurement pump cell 41 with respect to the input power. Accordingly, the inter-electrode distance in the measurement pump cell 41 being smaller than the inter-electrode distance in the main pump cell 21 enables the gas sensor S to realize highly accurate concentration measurement under both high and low concentrations.

### Features

As described above, the gas sensor S according to this embodiment includes the gas sensor element 100, the detection unit 111, and the temperature setting unit 112. The gas sensor element 100 is a sensor element constituted by six oxygen ion-conductive solid electrolyte layers. The gas sensor element 100 includes the internal space (the measurement target gas flow section 7) into which a measurement target gas is introduced, the measurement pump cell 41, and the heater 70 (heater unit), and further includes at least either the main pump cell 21 or the auxiliary pump cell 50 as the adjustment pump cell. The measurement pump cell 41 is an electrochemical pump cell constituted by the measurement electrode 44 provided in the measurement target gas flow section 7, the outer pump electrode 23 provided in a location different from the measurement target gas flow section 7, and solid electrolyte layers (the second solid electrolyte layer 6, the spacer layer 5, and the first solid electrolyte layer 4) that are present between the measurement electrode 44 and the outer pump electrode 23. The heater 70 is embedded in the gas sensor element 100 and heats the gas sensor element 100 to a specific temperature (sensor element drive temperature).

The adjustment pump cell included in the gas sensor element 100 is an electrochemical pump cell constituted by the inner pump electrode (the inner pump electrode 22 or the auxiliary pump electrode 51) facing the internal space (i.e., the measurement target gas flow section 7) in the gas sensor element 100, the outer pump electrode 23 or the third electrode in contact with at least one of the solid electrolyte layers 1 to 6 and exposed to the external space, and a solid electrolyte layer that is present between the inner pump electrode and the outer pump electrode 23 or the third electrode.

Specifically, the main pump cell 21 is an electrochemical pump cell constituted by the inner pump electrode 22 facing the measurement target gas flow section 7, the outer pump electrode 23, and the second solid electrolyte layer 6 held between the inner pump electrode 22 and the outer pump electrode 23. The auxiliary pump cell 50 is an electrochemical pump cell constituted by the auxiliary pump electrode 51, the outer pump electrode 23 (or an appropriate electrode on the outer side of the gas sensor element 100 that is in contact with at least one of the solid electrolyte layers 1 to 6), and a solid electrolyte layer (e.g., the second solid electrolyte layer 6) that is held therebetween. “An appropriate electrode on the outer side of the gas sensor element 100 that is in contact with at least one of the solid electrolyte layers 1 to 6” is also referred to as the third electrode.

The detection unit 111 detects a value of the cell resistance (impedance) of the adjustment pump cell included in the gas sensor element 100. That is, the detection unit 111 detects the value of the cell resistance of at least either the main pump cell 21 or the auxiliary pump cell 50.

The temperature setting unit 112 (adjustment unit) adjusts (sets) the specific temperature (sensor element drive temperature) such that the value of the cell resistance (impedance) of the adjustment pump cell that is detected by the detection unit 111 is the reference impedance 115 (predetermined value). That is, the temperature setting unit 112 sets the sensor element drive temperature such that the value of the cell resistance of at least either the main pump cell 21 or the auxiliary pump cell 50 is equal to the reference impedance 115.

In the gas sensor S, the slope of the cell resistance (the slope of the cell resistance with respect to the input power to the heater 70) of the adjustment pump cell is larger than the slope of the cell resistance of the measurement pump cell 41. That is, in the gas sensor S, the slope of the cell resistance (the slope of the cell resistance with respect to the input power to the heater 70) of at least either the main pump cell 21 or the auxiliary pump cell 50 is larger than the slope of the cell resistance of the measurement pump cell 41.

A gas sensor control method according to this embodiment is a method for controlling a gas sensor that includes the gas sensor element 100, and is an information processing method for executing a detection step and an adjustment step.

In the detection step, the value of the cell resistance (impedance) of the adjustment pump cell included in the gas sensor element 100 is detected. That is, in the detection step, the value of the cell resistance of at least either the main pump cell 21 or the auxiliary pump cell 50 is detected.

In the adjustment step, the specific temperature (sensor element drive temperature) is adjusted (set) such that the value of the cell resistance (impedance) of the adjustment pump cell that is detected in the detection step is the reference impedance 115 (predetermined value). That is, in the adjustment step, the sensor element drive temperature is set such that the value of the cell resistance of at least either the main pump cell 21 or the auxiliary pump cell 50 is equal to the reference impedance 115.

In the gas sensor control method according to this embodiment, the slope of the cell resistance (the slope of the cell resistance with respect to the input power to the heater 70) of the adjustment pump cell is larger than the slope of the cell resistance of the measurement pump cell 41. That is, in the gas sensor control method according to this embodiment, the slope of the cell resistance (the slope of the cell resistance with respect to the input power to the heater 70) of at least either the main pump cell 21 or the auxiliary pump cell 50 is larger than the slope of the cell resistance of the measurement pump cell 41.

In this configuration, the sensor element drive temperature is adjusted, i.e., controlled such that the value of the cell resistance of the adjustment pump cell (at least either the main pump cell 21 or the auxiliary pump cell 50) that is detected by the detection unit 111 (detection step) is the predetermined value. Also, the slope of the cell resistance of the adjustment pump cell with respect to the input power to the heater 70 is larger than the slope of the cell resistance of the measurement pump cell 41 with respect to the input power to the heater 70. In other words, as for the slope of the cell resistance with respect to the input power to the heater 70 in this configuration, the slope of the adjustment pump cell is larger than the slope of the measurement pump cell 41.

The following effects are achieved by controlling the sensor element drive temperature and setting the value of the cell resistance of the adjustment pump cell to the predetermined value. That is, the value of the cell resistance of the adjustment pump cell being kept at the predetermined value does not increase the resistance to the reaction of the adjustment pump cell, and eliminates the need to increase the pump voltage (i.e., at least either the pump voltage Vp0 or the voltage Vp1) to be applied to the adjustment pump cell. Accordingly, it is possible to avoid a situation where, as a result of increasing the pump voltage to be applied to the adjustment pump cell, an NOₓ specific gas in the measurement target gas is decomposed in the adjustment pump cell, resulting in deterioration of the measurement accuracy for the NOₓ concentration, particularly when the NOₓ concentration is high.

As for the slope of the cell resistance with respect to the input power to the heater 70, the slope of the adjustment pump cell is larger than the slope of the measurement pump cell 41. Therefore, a control can be performed with a small amount of input power such that the value of the cell resistance of the adjustment pump cell is the predetermined value.

Furthermore, since the slope of the cell resistance of the adjustment pump cell with respect to the input power to the heater 70 is large, the change in the sensor element drive temperature can be reduced when the input power (i.e., the sensor element drive temperature) is changed such that the value of the cell resistance of the adjustment pump cell is the predetermined value. The change in the offset value can be suppressed by reducing the change in the sensor element drive temperature, and highly accurate concentration measurement can be realized even when the NOₓ concentration in the measurement target gas is low.

In addition, as for the slope of the cell resistance with respect to the input power to the heater 70, the slope of the measurement pump cell 41 is small (compared with the slope of the adjustment pump cell). Therefore, the amount of change in the cell resistance of the measurement pump cell 41 is small even if the temperature (e.g., the temperature of the measurement pump cell 41) changes, and the change in the offset value can be suppressed.

As described above, the gas sensor S can avoid a situation where the measurement accuracy deteriorates when the NOₓ concentration in the measurement target gas is high, and also realize highly accurate concentration measurement when the NOₓ concentration in the measurement target gas is low. In other words, the gas sensor S can realize both highly accurate concentration measurement in environments where the NOₓ concentration in the measurement target gas is high and highly accurate concentration measurement in environments where the concentration is low.

In the gas sensor S, the slope of the cell resistance of the adjustment pump cell with respect to the input power to the heater 70 is 1.5 to 1000 times the slope of the cell resistance of the measurement pump cell 41 with respect to the input power to the heater 70. In this configuration, the slope of the cell resistance of the adjustment pump cell with respect to the input power to the heater 70 is 1.5 to 1000 times the slope of the cell resistance of the measurement pump cell 41 with respect to the input power to the heater 70. To realize highly accurate concentration measurement under both high and low concentrations, it is desirable to make the slope of the cell resistance of the adjustment pump cell with respect to the input power to the heater 70 larger than the slope of the cell resistance of the measurement pump cell 41 with respect to the input power to the heater 70, as mentioned above. The inventor confirmed through experiments that it is desirable to make the slope of the cell resistance of the adjustment pump cell with respect to the input power to the heater 70 1.5 to 1000 times the slope of the cell resistance of the measurement pump cell 41 with respect to the input power to the heater 70. Accordingly, the gas sensor S can realize highly accurate concentration measurement under both high and low concentrations by setting the slope of the cell resistance (the slope of the cell resistance with respect to the input power to the heater 70) of the adjustment pump cell to a value that is 1.5 to 1000 times the slope of the cell resistance of the measurement pump cell 41.

### Variations

Although an embodiment of the present invention has been described above, the description of the above embodiment is merely an illustration of the invention in all respects. Various improvements and variations may be made to the above embodiment. The constituent elements of the above embodiment may be omitted, replaced, and added as appropriate. The shape and dimensions of each constituent element of the above embodiment may be changed as appropriate, as per the mode of implementation. For example, the following changes are possible. Note that, in the following, the same constituent elements as those of the above embodiment are assigned the same reference numerals, and the description of the same features as the above embodiment is omitted as appropriate. The following variations can be combined as appropriate.

### (I) Configuration of Measurement Pump Cell and Main Pump Cell

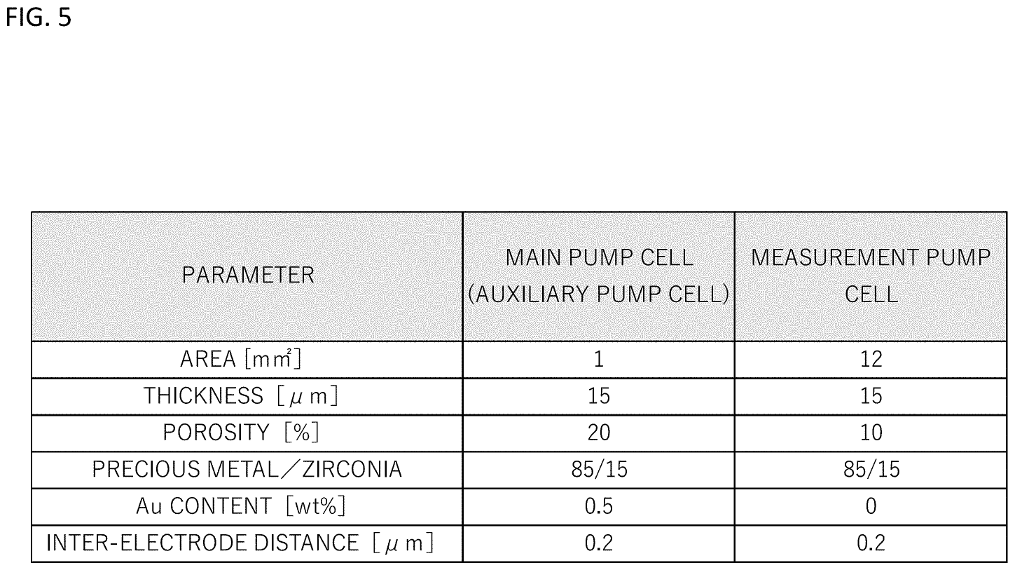

FIG. 5 shows examples of numerical values of the electrode area, thickness, porosity, ratio of precious metal to zirconia, and Au content, as well as the inter-electrode distance in the measurement pump cell 41 and the main pump cell 21 in a gas sensor S1 according to a variation.

### Regarding Electrode Area

In the gas sensor S1, the electrode area in the main pump cell 21, i.e., the area [mm²] of the inner pump electrode 22 (specifically, the face thereof that is exposed to the measurement target gas) is 1, as illustrated in FIG. 5. Meanwhile, the electrode area in the measurement pump cell 41, i.e., the area [mm²] of the measurement electrode 44 (specifically, the face thereof that is exposed to the measurement target gas) is 12. Therefore, in the gas sensor S1, the area of the measurement electrode 44 (the face thereof that is exposed to the measurement target gas) is larger than the area of the inner pump electrode 22 (the face thereof that is exposed to the measurement target gas).

As mentioned above, the slope of the cell resistance (the slope of the cell resistance with respect to the input power to the heater 70) of the main pump cell 21 can be made larger than the slope of the cell resistance of the measurement pump cell 41 by making the area of the measurement electrode 44 larger than the area of the inner pump electrode 22. By making the slope of the cell resistance of the main pump cell 21 larger than the slope of the cell resistance of the measurement pump cell 41, highly accurate concentration measurement can be realized in both environments where the NOₓ concentration in the measurement target gas is high and in environments where the concentration is low.

Accordingly, the area of the measurement electrode 44 being larger than the area of the inner pump electrode 22 enables the gas sensor S1 to realize highly accurate concentration measurement under both high and low concentrations.

### Regarding Electrode Thickness

In the gas sensor S1, the electrode thickness in the main pump cell 21, i.e., the thickness [μm] of the inner pump electrode 22 is 15, as illustrated in FIG. 5. Meanwhile, the electrode thickness in the measurement pump cell 41, i.e., the thickness [μm] of the measurement electrode 44 is 15. Therefore, in the gas sensor S1, the thickness of the measurement electrode 44 is the same as the thickness of the inner pump electrode 22.

As mentioned above, the slope of the cell resistance (the slope of the cell resistance with respect to the input power to the heater 70) of the main pump cell 21 can be made larger than the slope of the cell resistance of the measurement pump cell 41 by making the measurement electrode 44 thicker than the inner pump electrode 22. By making the slope of the cell resistance of the main pump cell 21 larger than the slope of the cell resistance of the measurement pump cell 41, highly accurate concentration measurement can be realized in both environments where the NOₓ concentration in the measurement target gas is high and in environments where the concentration is low.

Accordingly, making the measurement electrode 44 thicker than the inner pump electrode 22 enables the gas sensor S1 to realize highly accurate concentration measurement under both high and low concentrations.

### Regarding Electrode Porosity

In the gas sensor S1, the electrode porosity in the main pump cell 21, i.e., the porosity [%] of the inner pump electrode 22 is 20, as illustrated in FIG. 5. Meanwhile, the electrode porosity in the measurement pump cell 41, i.e., the porosity [%] of the measurement electrode 44 is 10. Therefore, in the gas sensor S1, the porosity of the measurement electrode 44 is lower than the porosity of the inner pump electrode 22.

As mentioned above, the slope of the cell resistance (the slope of the cell resistance with respect to the input power to the heater 70) of the main pump cell 21 can be made larger than the slope of the cell resistance of the measurement pump cell 41 by making the porosity of the measurement electrode 44 lower than the porosity of the inner pump electrode 22. By making the slope of the cell resistance of the main pump cell 21 larger than the slope of the cell resistance of the measurement pump cell 41, highly accurate concentration measurement can be realized in both environments where the NOₓ concentration in the measurement target gas is high and in environments where the concentration is low.

Accordingly, the porosity of the measurement electrode 44 being lower than the porosity of the inner pump electrode 22 enables the gas sensor S1 to realize highly accurate concentration measurement under both high and low concentrations.

### Regarding Ratio Between Precious Metal and Zirconia in Electrodes

In the gas sensor S1, both the inner pump electrode 22 and the measurement electrode 44 are cermet electrodes made of zirconia and precious metal. In the gas sensor S1, the ratio between precious metal and zirconia in the electrode of the main pump cell 21, i.e., the ratio between precious metal and zirconia in the inner pump electrode 22 is 85 (precious metal):15 (zirconia), as illustrated in FIG. 5. Meanwhile, the ratio between precious metal and zirconia in the electrode of the measurement pump cell 41, i.e., the ratio between precious metal and zirconia in the measurement electrode 44 is 85 (precious metal):15 (zirconia). Therefore, in the gas sensor S1, the ratio of precious metal to zirconia in the measurement electrode 44 is the same as the ratio of precious metal to zirconia in the inner pump electrode 22.

As for the ratio of precious metal to zirconia, the slope of the cell resistance (the slope of the cell resistance with respect to the input power to the heater 70) of the main pump cell 21 can be made larger than the slope of the cell resistance of the measurement pump cell 41 by making the ratio in the measurement electrode 44 higher than the ratio in the inner pump electrode 22, as mentioned above. By making the slope of the cell resistance of the main pump cell 21 larger than the slope of the cell resistance of the measurement pump cell 41, highly accurate concentration measurement can be realized in both environments where the NOₓ concentration in the measurement target gas is high and in environments where the concentration is low.

Accordingly, the ratio of precious metal to zirconia in the measurement electrode 44 being higher than the ratio in the inner pump electrode 21 enables the gas sensor S1 to realize highly accurate concentration measurement under both high and low concentrations.

### Regarding Au Content in Electrodes

In the gas sensor S1, the Au content in the electrode of the main pump cell 21, i.e., the Au content [wt %] in the inner pump electrode 22 is 0.5, as illustrated in FIG. 5. Meanwhile, the Au content in the electrode of the measurement pump cell 41, i.e., the Au content [wt %] in the measurement electrode 44 is 0. Therefore, in the gas sensor S1, the Au content in the measurement electrode 44 is lower than the Au content in the inner pump electrode 22.

As mentioned above, the slope of the cell resistance (the slope of the cell resistance with respect to the input power to the heater 70) of the main pump cell 21 can be made larger than the slope of the cell resistance of the measurement pump cell 41 by making the Au content in the measurement electrode 44 lower than the Au content in the inner pump electrode 22. By making the slope of the cell resistance of the main pump cell 21 larger than the slope of the cell resistance of the measurement pump cell 41, highly accurate concentration measurement can be realized in both environments where the NOₓ concentration in the measurement target gas is high and in environments where the concentration is low.

Accordingly, the Au content in the measurement electrode 44 being lower than the Au content in the inner pump electrode 22 enables the gas sensor S1 to realize highly accurate concentration measurement under both high and low concentrations.

### Regarding Inter-Electrode Distance

In the gas sensor S1, the inter-electrode distance in the main pump cell 21, i.e., the distance [μm] between the inner pump electrodes 22 and the outer pump electrode 23 is 0.2, as illustrated in FIG. 5. Meanwhile, the inter-electrode distance in the measurement pump cell 41, i.e., the distance [μm] between the measurement electrode 44 and the outer pump electrode 23 is 0.2. Therefore, in the gas sensor S1, the inter-electrode distance in the measurement pump cell 41 (the distance between the measurement electrode 44 and the outer pump electrode 23) is the same as the inter-electrode distance in the main pump cell 21 (the distance between the inner pump electrode 22 and the outer pump electrode 23).

As mentioned above, the slope of the cell resistance (the slope of the cell resistance with respect to the input power to the heater 70) of the main pump cell 21 can be made larger than the slope of the cell resistance of the measurement pump cell 41 by making the inter-electrode distance in the measurement pump cell 41 smaller (shorter) than the inter-electrode distance in the main pump cell 21. By making the slope of the cell resistance of the main pump cell 21 larger than the slope of the cell resistance of the measurement pump cell 41, highly accurate concentration measurement can be realized in both environments where the NOₓ concentration in the measurement target gas is high and in environments where the concentration is low.

Accordingly, the inter-electrode distance in the measurement pump cell 41 being smaller than the inter-electrode distance in the main pump cell 21 enables the gas sensor S1 to realize highly accurate concentration measurement under both high and low concentrations.

### Notes on Electrode Area

The above description is of an example where the main pump cell 21 is an adjustment pump cell whose electrode area is smaller than the area of the electrode (measurement electrode 44) of the measurement pump cell 41. That is, the above description is of an example where the slope of the cell resistance of the main pump cell 21 serving as the adjustment pump cell can be made larger than the slope of the cell resistance of the measurement pump cell 41 by making the area of the measurement electrode 44 larger than the area of the inner pump electrode 22. However, the auxiliary pump cell 50 may alternatively be an adjustment pump cell whose electrode area is smaller than the area of the electrode (measurement electrode 44) of the measurement pump cell 41. That is, the area of the measurement electrode 44 may be larger than the area of the auxiliary pump electrode 51 of the auxiliary pump cell 50.

Further, the areas of the electrodes of both the main pump cell 21 and the auxiliary pump cell 50 may be smaller than the area of the electrode of the measurement pump cell 41. That is, both the area of the inner pump electrode 22 and the area of the auxiliary pump electrode 51 of the auxiliary pump cell 50 may be smaller than the area of the measurement electrode 44.

In a gas sensor according to one aspect of the present invention, the area of the measurement electrode 44 need only be larger than the area of the electrode of at least either the main pump cell 21 or the auxiliary pump cell 50 (e.g., the area of at least either the inner pump electrode 22 or the auxiliary pump electrode 51).

The slope of the cell resistance (the slope of the cell resistance with respect to the input power to the heater 70) of the adjustment pump cell can be made larger than the slope of the cell resistance of the measurement pump cell 41 by making the area of the electrode of the measurement pump cell 41 larger than the area of the electrode of the adjustment pump cell. By making the slope of the cell resistance of the adjustment pump cell larger than the slope of the cell resistance of the measurement pump cell 41, highly accurate concentration measurement can be realized in both environments where the NOₓ concentration in the measurement target gas is high and environments where the concentration is low.

### Notes on Electrode Thickness

The above description is of an example where the main pump cell 21 is an adjustment pump cell having an electrode that is thinner than the electrode (measurement electrode 44) of the measurement pump cell 41. That is, the above description is of an example where the slope of the cell resistance of the main pump cell 21 serving as the adjustment pump cell can be made larger than the slope of the cell resistance of the measurement pump cell 41 by making the measurement electrode 44 thicker than the inner pump electrode 22. However, the auxiliary pump cell 50 may alternatively be an adjustment pump cell having an electrode that is thinner than the electrode (measurement electrode 44) of the measurement pump cell 41. That is, the measurement electrode 44 may be thicker than the auxiliary pump electrode 51 of the auxiliary pump cell 50.

Further, the electrodes of both the main pump cell 21 and the auxiliary pump cell 50 may be thinner than the electrode of the measurement pump cell 41. That is, both the inner pump electrode 22 and the auxiliary pump electrode 51 of the auxiliary pump cell 50 may be thinner than the measurement electrode 44.

In a gas sensor according to one aspect of the present invention, the measurement electrode 44 need only be thicker than the electrode of at least either the main pump cell 21 or the auxiliary pump cell 50 (e.g., the thickness of at least either the inner pump electrode 22 or the auxiliary pump electrode 51).

The slope of the cell resistance (the slope of the cell resistance with respect to the input power to the heater 70) of the adjustment pump cell can be made larger than the slope of the cell resistance of the measurement pump cell 41 by making the electrode of the measurement pump cell 41 thicker than the electrode of the adjustment pump cell. By making the slope of the cell resistance of the adjustment pump cell larger than the slope of the cell resistance of the measurement pump cell 41, highly accurate concentration measurement can be realized in both environments where the NOₓ concentration in the measurement target gas is high and environments where the concentration is low.

### Notes on Electrode Porosity

The above description is of an example where the main pump cell 21 is an adjustment pump cell having an electrode whose porosity is higher than the porosity of the electrode (measurement electrode 44) of the measurement pump cell 41. That is, the above description is of an example where the slope of the cell resistance of the main pump cell 21 serving as the adjustment pump cell can be made larger than the slope of the cell resistance of the measurement pump cell 41 by making the porosity of the measurement electrode 44 lower than the porosity of the inner pump electrode 22. However, the auxiliary pump cell 50 may alternatively be an adjustment pump cell having an electrode whose porosity is higher than the porosity of the electrode (measurement electrode 44) of the measurement pump cell 41. That is, the porosity of the measurement electrode 44 may be lower than the porosity of the auxiliary pump electrode 51 of the auxiliary pump cell 50.

Further, the porosities of the electrodes of both the main pump cell 21 and the auxiliary pump cell 50 may be higher than the porosity of the electrode of the measurement pump cell 41. That is, both the porosity of the inner pump electrode 22 and the porosity of the auxiliary pump electrode 51 of the auxiliary pump cell 50 may be higher than the porosity of the measurement electrode 44.

In a gas sensor according to one aspect of the present invention, the porosity of the measurement electrode 44 need only be lower than the porosity of the electrode of at least either the main pump cell 21 or the auxiliary pump cell 50 (e.g., the porosity of at least either the inner pump electrode 22 or the auxiliary pump electrode 51).

The slope of the cell resistance (the slope of the cell resistance with respect to the input power to the heater 70) of the adjustment pump cell can be made larger than the slope of the cell resistance of the measurement pump cell 41 by making the porosity of the electrode of the measurement pump cell 41 lower than the porosity of the electrode of the adjustment pump cell. By making the slope of the cell resistance of the adjustment pump cell larger than the slope of the cell resistance of the measurement pump cell 41, highly accurate concentration measurement can be realized in both environments where the NOₓ concentration in the measurement target gas is high and environments where the concentration is low.

### Notes on Ratio Between Precious Metal and Zirconia

The above description is of an example where, as for the ratio of precious metal to zirconia, the main pump cell 21 is an adjustment pump cell having a cermet electrode with a ratio lower than the ratio in the electrode (measurement electrode 44) of the measurement pump cell 41. That is, the above description is of an example where, as for the ratio of precious metal to zirconia, the slope of the cell resistance of the main pump cell 21 serving as the adjustment pump cell can be made larger than the slope of the cell resistance of the measurement pump cell 41 by making the ratio in the measurement electrode 44 higher than the ratio in the inner pump electrode 22. However, as for the ratio of precious metal to zirconia, the auxiliary pump cell 50 may be an adjustment pump cell having a cermet electrode with a ratio lower than the ratio in the electrode (measurement electrode 44) in the measurement pump cell 41. That is, as for the ratio of precious metal to zirconia, the ratio in the measurement electrode 44 may be higher than the ratio in the auxiliary pump electrode 51 of the auxiliary pump cell 50.

Further, as for the ratio of precious metal to zirconia, the ratios in the electrodes of both the main pump cell 21 and the auxiliary pump cell 50 may be lower than the ratio in the electrode of the measurement pump cell 41. That is, as for the ratio of precious metal to zirconia, both the ratio in the inner pump electrode 22 and the ratio in the auxiliary pump electrode 51 of the auxiliary pump cell 50 may be lower than the ratio in the measurement electrode 44.

In a gas sensor according to one aspect of the present invention, as for the ratio of precious metal to zirconia, the ratio in the measurement electrode 44 need only be higher than the ratio in the electrode of at least either the main pump cell 21 or the auxiliary pump cell 50 (e.g., the ratio in at least either the inner pump electrode 22 or the auxiliary pump electrode 51).

As for the ratio of precious metal to zirconia, the slope of the cell resistance (the slope of the cell resistance with respect to the input power to the heater 70) of the adjustment pump cell can be made larger than the slope of the cell resistance of the measurement pump cell 41 by making the ratio in the electrode of the measurement pump cell 41 higher than the ratio in the electrode of the adjustment pump cell. By making the slope of the cell resistance of the adjustment pump cell larger than the slope of the cell resistance of the measurement pump cell 41, highly accurate concentration measurement can be realized in both environments where the NOₓ concentration in the measurement target gas is high and environments where the concentration is low.

### Notes on Au Content

The above description is of an example where the main pump cell 21 is an adjustment pump cell having an electrode whose Au content is higher than the Au content in the electrode (measurement electrode 44) of the measurement pump cell 41. That is, the above description is of an example where the slope of the cell resistance of the main pump cell 21 serving as the adjustment pump cell can be made larger than the slope of the cell resistance of the measurement pump cell 41 by making the Au content in the measurement electrode 44 lower than the Au content in the inner pump electrode 22. However, the auxiliary pump cell 50 may alternatively be an adjustment pump cell having an electrode whose Au content is higher than the Au content in the electrode (measurement electrode 44) of the measurement pump cell 41. That is, the Au content in the measurement electrode 44 may be lower than the Au content in the auxiliary pump electrode 51 of the auxiliary pump cell 50.

Further, the Au contents in the electrodes of both the main pump cell 21 and the auxiliary pump cell 50 may be higher than the Au content in the electrode of the measurement pump cell 41. That is, both the Au content in the inner pump electrode 22 and the Au content in the auxiliary pump electrode 51 of the auxiliary pump cell 50 may be higher than the Au content in the measurement electrode 44.

In a gas sensor according to one aspect of the present invention, the Au content in the measurement electrode 44 need only be lower than the Au content in the electrode of at least either the main pump cell 21 or the auxiliary pump cell 50 (e.g., the Au content in at least either the inner pump electrode 22 or the auxiliary pump electrode 51).

The slope of the cell resistance (the slope of the cell resistance with respect to the input power to the heater 70) of the adjustment pump cell can be made larger than the slope of the cell resistance of the measurement pump cell 41 by making the Au content in the electrode of the measurement pump cell 41 lower than the Au content in the electrode of the adjustment pump cell. By making the slope of the cell resistance of the adjustment pump cell larger than the slope of the cell resistance of the measurement pump cell 41, highly accurate concentration measurement can be realized in both environments where the NOₓ concentration in the measurement target gas is high and environments where the concentration is low.

### Notes on Inter-Electrode Distance

The above description is of an example where the main pump cell 21 is an adjustment pump cell having an inter-electrode distance that is larger (longer) than the inter-electrode distance in the measurement pump cell 41. That is, the above description is of an example where the slope of the cell resistance of the main pump cell 21 serving as the adjustment pump cell can be made larger than the slope of the cell resistance of the measurement pump cell 41 by making the inter-electrode distance in the measurement pump cell 41 (the distance between the measurement electrode 44 and the outer pump electrode 23) smaller (shorter) than the inter-electrode distance in the main pump cell 21 (the distance between the inner pump electrode 22 and the outer pump electrode 23). However, the auxiliary pump cell 50 may alternatively be an adjustment pump cell having an inter-electrode distance that is larger than the inter-electrode distance in the electrode in the measurement pump cell 41. That is, the inter-electrode distance in the measurement pump cell 41 may be smaller than the inter-electrode distance in the auxiliary pump cell 50 (the distance between the auxiliary pump electrode 51 and the outer pump electrode 23 or the third electrode of the auxiliary pump cell 50).

Further, the inter-electrode distances in both the main pump cell 21 and the auxiliary pump cell 50 may be larger than the inter-electrode distance in the measurement pump cell 41. That is, both the distance between the inner pump electrode 22 and the outer pump electrode 23 and the distance between the auxiliary pump electrode 51 and the outer pump electrode 23 or the third electrode may be larger than the distance between the measurement electrode 44 and the outer pump electrode 23.

In a gas sensor according to one aspect of the present invention, the inter-electrode distance in the measurement pump cell 41 need only be smaller than the inter-electrode distance in at least either the main pump cell 21 or the auxiliary pump cell 50.

The slope of the cell resistance (the slope of the cell resistance with respect to the input power to the heater 70) of the adjustment pump cell can be made larger than the slope of the cell resistance of the measurement pump cell 41 by making the inter-electrode distance in the measurement pump cell 41 smaller than the inter-electrode distance in the adjustment pump cell. By making the slope of the cell resistance of the adjustment pump cell larger than the slope of the cell resistance of the measurement pump cell 41, highly accurate concentration measurement can be realized in both environments where the NOₓ concentration in the measurement target gas is high and environments where the concentration is low.

The above description is of the following conditions 1 to 6 as conditions regarding the configuration of the measurement pump cell 41 and the adjustment pump cell for making the slope of the cell resistance (the slope of the cell resistance with respect to the input power to the heater 70) of the adjustment pump cell larger than the slope of the cell resistance of the measurement pump cell 41. That is, the condition 1 is that the electrode area in the measurement pump cell 41 is larger than the electrode area in the adjustment pump cell. The condition 2 is that the electrode of the measurement pump cell 41 is thicker than the electrode of the adjustment pump cell. The condition 3 is that the electrode porosity in the measurement pump cell 41 is lower than the electrode porosity in the adjustment pump cell. The condition 4 is that the ratio of precious metal to zirconia in the electrode of the measurement pump cell 41 is higher than the ratio of precious metal to zirconia in the electrode of the adjustment pump cell. The condition 5 is that the Au content in the electrode of the measurement pump cell 41 is lower than the Au content in the electrode of the adjustment pump cell. The condition 6 is that the inter-electrode distance in the measurement pump cell 41 is smaller (shorter) than the inter-electrode distance in the adjustment pump cell.

It is not essential that all of the conditions 1 to 6 are satisfied in order to make the slope of the cell resistance (the slope of the cell resistance with respect to the input power to the heater 70) of the adjustment pump cell larger than the slope of the cell resistance of the measurement pump cell 41. The inventor confirmed through experiments that it is effective to configure the adjustment pump cell and the measurement pump cell 41 such that at least one of the conditions 1 to 6 is satisfied in order to make the slope of the cell resistance (the slope of the cell resistance with respect to the input power to the heater 70) of the adjustment pump cell larger than the slope of the cell resistance of the measurement pump cell 41. Note that, in the aforementioned experiments, the inventor has also confirmed that, to suppress the change in the offset value that affects the measurement accuracy, it is desirable that the slope of the cell resistance (the slope of the cell resistance with respect to the input power to the heater 70) of the adjustment pump cell takes a value that is 1.5 to 1000 times the slope of the cell resistance of the measurement pump cell 41. The experiments are described in detail below.

### (II) Others

In the above embodiment, the laminate of the gas sensor element 100 is constituted by six solid electrolyte layers. However, the number of solid electrolyte layers to constitute the laminate need not be limited to this example, and may be selected as appropriate, as per the mode of implementation.

In the above embodiment, the internal space (i.e., the measurement target gas flow section 7) into which the measurement target gas is introduced is located at a position demarcated by the first solid electrolyte layer 4, the spacer layer 5, and the second solid electrolyte layer 6. However, the location of the measurement target gas flow section 7 need not be limited to this example, and may be selected as appropriate, as per the mode of implementation. The orientations of a first face, a second face, a first pump electrode, a second pump electrode, a first lead, and a second lead may be selected as appropriate in accordance with the configuration of the laminate and the internal space.

In the above embodiment, the measurement target gas flow section 7 has a three-cavity structure. However, the configuration of the measurement target gas flow section 7 need not be limited to this example, and may be selected as appropriate, as per the mode of implementation. In another example, the fourth diffusion control portion 18 and the third internal cavity 19 may be omitted, and thus, the measurement target gas flow section 7 may have a two-cavity structure. In this case, the measurement electrode 44 may be provided at a position separated from the third diffusion control portion 16 on the upper face of the first solid electrolyte layer 4 adjacent to the second internal cavity 17. That is, the measurement target gas flow section 7 may include two hollow spaces into or from which oxygen is pumped, or may include only one such hollow space. In addition, it is not essential, either, for the gas sensor element 100 to include one or more diffusion control portions.

In FIG. 1, both the inner pump electrode 22 and the outer pump electrode 23 are exposed to a space. However, the manner of adjoining a space need not be limited to this mode, and may alternatively be indirectly adjoined via a coating or the like. As another example, the outer pump electrode 23 may be coated with a protection member or the like.

In the above embodiment, the reference gas inlet space 43 is provided. However, the configuration of the gas sensor element 100 need not be limited to this example. In another example, the first solid electrolyte layer 4 may extend to the rear end of the gas sensor element 100, and the reference gas inlet space 43 may be omitted. In this case, the atmosphere inlet layer 48 may extend to the rear end of the gas sensor element 100.

In the above embodiment, the gas sensor element 100 is configured to measure the concentration of nitrogen oxide (NOₓ). However, the gas sensor element of the present invention need not be limited to such a gas sensor element configured to measure the NOₓ concentration. In another example, the gas sensor element of the present invention may be another gas sensor element, such as a gas sensor element configured to measure the oxygen concentration. For example, a gas sensor element for measuring the oxygen concentration can be configured by omitting the auxiliary pump cell and the measurement pump cell and disposing the reference electrode below the main pump electrode in the gas sensor element 100 according to the above embodiment. In this case, the gas sensor element can measure the oxygen concentration in the measurement target gas by pumping out oxygen using the main pump cell.

### EXAMPLES

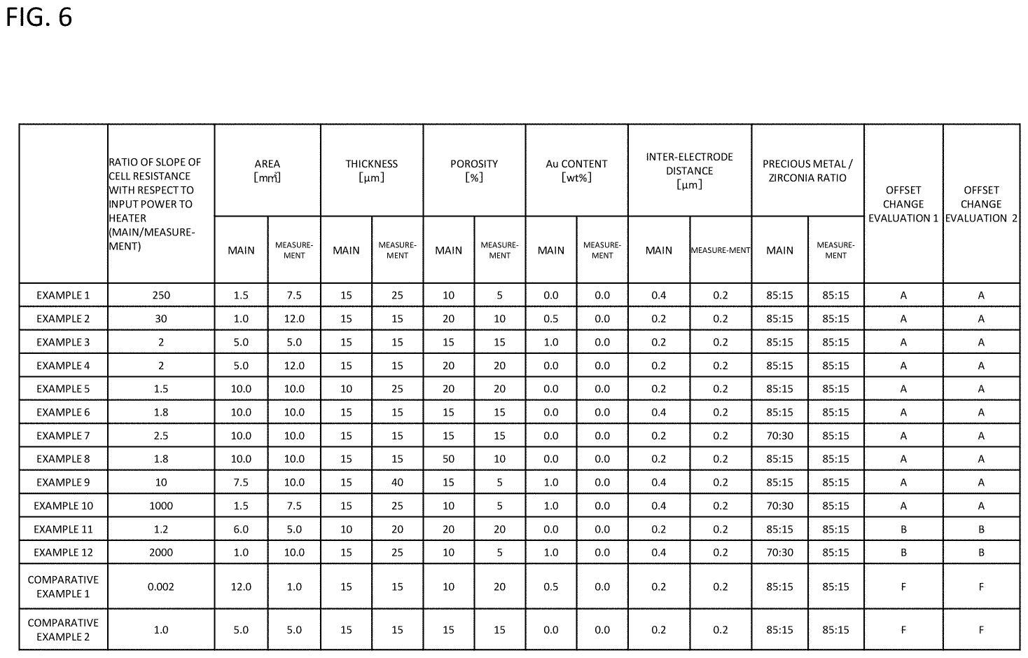

FIG. 6 shows an organized summary of the experiments implemented by the inventor. Specifically, FIG. 6 shows numerical values of various parameters and confirmed results regarding gas sensors according to examples 1 to 12, and comparative examples 1 and 2 with which the inventor checked the amount of change in the offset value (offset change rate). As will be described below, the inventor confirmed the results of evaluation 1 (offset change evaluation 1) and evaluation 2 (offset change evaluation 2) regarding the amount of change in the offset value in the gas sensors according to the examples 1 to 12 and the comparative examples 1 and 2. As mentioned above, the change in the offset value affects the measurement accuracy of the gas sensor. The less the offset value changes, the less the impact on the measurement accuracy of the gas sensor is, and the less likely the measurement accuracy is to deteriorate. Note that all of the gas sensors according to the examples 1 to 12 and the comparative examples 1 and 2 adopt the configuration show in FIG. 1.

The result of the offset change evaluation 1 was A for the gas sensors according to the examples 1 to 10 where the slope of the cell resistance of the adjustment pump cell took a value that was 1.5 to 1000 times the slope of the cell resistance of the measurement pump cell 41, as shown in FIG. 6. Although the details will be described later, the offset change evaluation 1 refers to the evaluation of the amount of change in the offset value before and after a change in the gas temperature, and A represents an evaluation result indicating “remarkably good”. For the gas sensors according to the examples 1 to 10, the offset change evaluation 2, which indicates the amount of change in the offset value before and after the implementation of a durability test (the details of which will be described later), was also A. Meanwhile, the result of the offset change evaluation 1 was B for the gas sensors according to the examples 11 and 12 where the slope of the cell resistance of the adjustment pump cell was not in the range from 1.5 to 1000 times the slope of the cell resistance of the measurement pump cell 41, but the former was larger than the latter. B represents the next best evaluation result after A, i.e., “good”. For the gas sensors according to the examples 11 and 12, the result of the offset change evaluation 2 was also B. In contrast, the result of the offset change evaluation 1 was F for the gas sensors in the comparative examples 1 and 2 where the slope of the cell resistance of the adjustment pump cell was smaller than or the same as the slope of the cell resistance of the measurement pump cell 41. F represents the worst evaluation result, i.e., “poor”. For the gas sensors in the comparative examples 1 and 2, the result of the offset change evaluation 2 was also B.

Thus, the inventor confirmed through the experiments shown in FIG. 6 that the change in the offset value can be suppressed by making the slope of the cell resistance of the adjustment pump cell larger than the slope of the cell resistance of the measurement pump cell 41. Specifically, the inventor confirmed through the experiments shown in FIG. 6 that the change in the offset value can be effectively suppressed by setting the slope of the cell resistance of the adjustment pump cell to a value that is 1.5 to 1000 times the slope of the cell resistance of the measurement pump cell 41. Further, the inventor confirmed through the experiments shown in FIG. 6 that it is desirable to configure the measurement pump cell 41 and the adjustment pump cell so as to satisfy at least one of the aforementioned conditions 1 to 6 in order to make the slope of the cell resistance of the adjustment pump cell with respect to the input power to the heater 70 larger than the slope of the cell resistance of the measurement pump cell 41 with respect to the input power to the heater 70. The content shown in FIG. 6 will be described in detail below.

Note that FIG. 6 shows examples where the main pump cell 21 is the adjustment pump cell. That is, in FIG. 6, the condition 1 means the condition that the area of the measurement electrode 44 is larger than the area of the inner pump electrode 22. The condition 2 means the condition that the measurement electrode 44 is thicker than the inner pump electrode 22. The condition 3 means the condition that the porosity of the measurement electrode 44 is lower than the porosity of the inner pump electrode 22. The condition 4 means the condition that the ratio of precious metal to zirconia in the measurement electrode 44 is higher than the ratio of precious metal to zirconia in the inner pump electrode 22. The condition 5 means the condition that the Au content in the measurement electrode 44 is lower than the Au content in the inner pump electrode 22. The condition 6 means the condition that the distance between the measurement electrode 44 and the outer pump electrode 23 is smaller (shorter) than the distance between the inner pump electrode 22 and the outer pump electrode 23.

In the table in FIG. 6, “RATIO OF SLOPE OF CELL RESISTANCE WITH RESPECT TO INPUT POWER TO HEATER (MAIN/MEASUREMENT)” indicates the ratio (multiplying factor) of the slope of the cell resistance of the main pump cell 21 with respect to the input power to the heater 70 to the slope of the cell resistance of the measurement pump cell 41 with respect to the input power to the heater 70. “MAIN” below “AREA [mm²]” indicates the electrode area in the main pump cell 21, i.e., the area [mm²] of the inner pump electrode 22 (specifically, of the face thereof exposed to the measurement target gas). “MEASUREMENT” below “AREA [mm²]” indicates the electrode area in the measurement pump cell 41, i.e., the area [mm²] of the measurement electrode 44 (specifically, of the face thereof exposed to the measurement target gas). “MAIN” below “THICKNESS [μm]” indicates the electrode thickness in the main pump cell 21, i.e., the thickness [μm] of the inner pump electrode 22. “MEASUREMENT” below “THICKNESS [μm]” indicates the electrode thickness in the measurement pump cell 41, i.e., the thickness [μm] of the measurement electrode 44. “MAIN” below “POROSITY [%]” indicates the electrode porosity of the main pump cell 21, i.e., the porosity [%] of the inner pump electrode 22. “MEASUREMENT” below “POROSITY [%]” indicates the electrode porosity of the measurement pump cell 41, i.e., the porosity [%] of the measurement electrode 44. “MAIN” below “Au CONTENT [wt %]” indicates the Au content in the electrode of the main pump cell 21, i.e., the Au content [wt %] in the inner pump electrode 22. “MEASUREMENT” below “Au CONTENT [wt %]” indicates the Au content in the electrode of the measurement pump cell 41, i.e., the Au content [wt %] of the measurement electrode 44. “MAIN” below “INTER-ELECTRODE DISTANCE [μm]” indicates the inter-electrode distance in the main pump cell 21, i.e., the distance [μm] between the inner pump electrode 22 and the outer pump electrode 23. “MEASUREMENT” below “INTER-ELECTRODE DISTANCE [μm]” indicates the inter-electrode distance in the measurement pump cell 41, i.e., the distance [μm] between the measurement electrode 44 and the outer pump electrode 23. “MAIN” below “PRECIOUS METAL/ZIRCONIA RATIO” indicates the ratio between precious metal and zirconia in the electrode of the main pump cell 21, i.e., the ratio between precious metal and zirconia in the inner pump electrode 22. “MEASUREMENT” below “PRECIOUS METAL/ZIRCONIA RATIO” indicates the ratio between precious metal and zirconia in the electrode of the measurement pump cell 41, i.e., the ratio between precious metal and zirconia in the measurement electrode 44.

“OFFSET CHANGE EVALUATION 1” and “OFFSET CHANGE EVALUATION 2” indicate the magnitude of changes in the offset value (offset change) in the gas sensors according to the examples 1 to 12 and the comparative examples 1 and 2.

Specifically, the offset change evaluation 1 indicates the magnitude of an offset change in NOₓ output (e.g., the pump current Ip2) of each gas sensor when the gas temperature is changed from 100° C. to 400° C. in an environment where NOₓ is 0 ppm and O₂ is 18%. The result A of the offset change evaluation 1 indicates that the offset change in the NOₓ output before and after the change in the gas temperature was within ±7 ppm. The result B of the offset change evaluation 1 indicates that the offset change in the NOₓ output before and after the change in the gas temperature was more than ±7 ppm and within ±15 ppm. The result F of the offset change evaluation 1 indicates that the offset change in the NOₓ output before and after the change in the gas temperature was more than ±15 ppm.

The offset change evaluation 2 indicates the magnitude of an offset change (the amount of change in the offset value) in the gas sensors according to the examples 1 to 12 and the comparative examples 1 and 2 before and after the implementation of the following durability test. That is, the inventor implemented, as the aforementioned durability test, a test in which each of the gas sensors was attached to an exhaust gas pipe of an automobile, and a 40-minute driving pattern was repeated for 2000 hours in a range where the engine speed was 1500 to 3500 rpm and the load torque was 0 to 350 N·m. Note that, in this durability test, the gas temperature ranged from 200 to 600 degrees Celsius, and the NOₓ concentration ranged from 0 to 1500 ppm. For each gas sensor, the offset value was measured when a model gas with a NOₓ concentration of 0 ppm, an O₂ concentration of 0%, and an H₂O concentration of 3% was caused to flow before and after the implementation of the aforementioned durability test, and the amount of change in the offset value was examined. The result A of the offset change evaluation 2 indicates that the offset change in the NOₓ output before and after the implementation of the aforementioned durability test was within ±7 ppm. The result B of the offset change evaluation 2 indicates that the offset change in the NOₓ output before and after the implementation of the aforementioned durability test was more than ±7 ppm and within ±15 ppm. The result F of the offset change evaluation 2 indicates that the offset change in the NOₓ output before and after the implementation of the aforementioned durability test was more than ±15 ppm.

As mentioned above, the smaller the offset change in the NOₓ output is, the smaller the impact on the measurement accuracy of the gas sensors is, and the less likely the measurement accuracy is to deteriorate. The offset change evaluation 1 and the offset change evaluation 2 are evaluations of the amount of change in the offset value in the gas sensors according to the examples 1 to 12 and the comparative examples 1 and 2 before and after the respective conditions (the gas temperature and the implementation of the durability test) were changed. A method of correcting a fixed value every certain time, that is, time correction can be used before and after such a change in the conditions. Therefore, if the amount of change in the offset value before and after the conditions are changed is within the range from −15% to +15%, the aforementioned time correction can be effectively used, and it can be evaluated that the change in the offset value has been suppressed. Thus, if the amount of change in the offset value before and after the conditions were changed was within ±7 ppm in the offset change evaluations 1 and 2, this result was evaluated as A (remarkably good). Similarly, if the amount of change in the offset value before and after the conditions were changed was more than ±7 ppm and within ±15 ppm, this result was evaluated as B (good). If the amount of change in the offset value before and after the conditions were changed was more than ±15 ppm, this result was evaluated as F (poor). In the following description, the units are omitted to simplify the description.

### Regarding Example 1

The electrode area in the main pump cell 21 (“MAIN” below “AREA”) in the example 1 is 1.5, and the electrode area in the measurement pump cell 41 (“MEASUREMENT” below “AREA”) is 7.5. In other words, in the example 1, the electrode area in the measurement pump cell 41 (the area of the measurement electrode 44) is larger than the electrode area in the main pump cell 21 (the area of the inner pump electrode 22), and satisfies the condition 1. The electrode thickness in the main pump cell 21 (“MAIN” below “THICKNESS”) in the example 1 is 15, and the electrode thickness in the measurement pump cell 41 (“MEASUREMENT” below “THICKNESS”) is 25. In other words, in the example 1, the electrode of the measurement pump cell 41 (the measurement electrode 44) is thicker than the electrode of the main pump cell 21 (the inner pump electrode 22), and satisfies the condition 2. The electrode porosity in the main pump cell 21 (“MAIN” below “POROSITY”) in the example 1 is 10, and the electrode porosity in the measurement pump cell 41 (“MEASUREMENT” below “POROSITY”) is 5. In other words, in the example 1, the electrode porosity in the measurement pump cell 41 (the porosity of the measurement electrode 44) is lower than the electrode porosity in the main pump cell 21 (the porosity of the inner pump electrode 22), and satisfies the condition 3. The Au content in the electrode of the main pump cell 21 (“MAIN” below “Au CONTENT”) in the example 1 is 0.0, and the Au content in the electrode of the measurement pump cell 41 (“MEASUREMENT” below “Au CONTENT”) is 0.0. In other words, in the example 1, the Au content in the electrode of the measurement pump cell 41 (the Au content in the measurement electrode 44) is equal to the Au content in the electrode of the main pump cell 21 (the Au content in the inner pump electrode 22), and does not satisfy the condition 5. The inter-electrode distance in the main pump cell 21 (“MAIN” below “INTER-ELECTRODE DISTANCE”) in the example 1 is 0.4, and the inter-electrode distance in the measurement pump cell 41 (“MEASUREMENT” below “INTER-ELECTRODE DISTANCE”) is 0.2. In other words, in the example 1, the inter-electrode distance in the measurement pump cell 41 (the distance between the measurement electrode 44 and the outer pump electrode 23) is smaller than the inter-electrode distance in the main pump cell 21 (the distance between the inner pump electrode 22 and the outer pump electrode 23), and satisfies the condition 6. The ratio between precious metal and zirconia in the electrode of the main pump cell 21 (“MAIN” below “PRECIOUS METAL/ZIRCONIA RATIO”) in the example 1 is 85:15, and the ratio between precious metal and zirconia in the electrode of the measurement pump cell 41 (“MEASUREMENT” below “PRECIOUS METAL/ZIRCONIA RATIO”) is 85:15. In other words, in the example 1, the ratio of precious metal to zirconia in the electrode of the measurement pump cell 41 (the ratio of precious metal to zirconia in the measurement electrode 44) is equal to the ratio of precious metal to zirconia in the electrode of the main pump cell 21 (the ratio of precious metal to zirconia in the inner pump electrode 22), and does not satisfy the condition 4.

Accordingly, of the aforementioned conditions 1 to 6, the conditions 4 and 5 are not satisfied but the conditions 1, 2, 3, and 6 are satisfied in the example 1. Also, “RATIO OF SLOPE OF CELL RESISTANCE TO INPUT POWER TO HEATER (MAIN/MEASUREMENT)” in the example 1 is 250. In other words, in the gas sensor according to the example 1, the slope of the cell resistance of the main pump cell 21 (the slope of the cell resistance with respect to the input power to the heater 70) takes a value that is 1.5 to 1000 times the slope of the cell resistance of the measurement pump cell 41. The results of the offset change evaluations 1 and 2 for the gas sensor according to the example 1 were both A, i.e., the best evaluation result.

### Regarding Example 2

The electrode area in the main pump cell 21 in the example 2 is 1.0, and the electrode area in the measurement pump cell 41 is 12.0. In other words, in the example 2, the electrode area in the measurement pump cell 41 (the area of the measurement electrode 44) is larger than the electrode area in the main pump cell 21 (the area of the inner pump electrode 22), and satisfies the condition 1. The electrode thickness in the main pump cell 21 in the example 2 is 15, and the electrode thickness in the measurement pump cell 41 is 15. In other words, in the example 2, the electrode thickness in the measurement pump cell 41 (the thickness of the measurement electrode 44) is equal to the electrode thickness in the main pump cell 21 (the thickness of the inner pump electrode 22), and does not satisfy the condition 2. The electrode porosity in the main pump cell 21 in the example 2 is 20, and the electrode porosity in the measurement pump cell 41 is 10. In other words, in the example 2, the electrode porosity in the measurement pump cell 41 (the porosity of the measurement electrode 44) is lower than the electrode porosity in the main pump cell 21 (the porosity of the inner pump electrode 22), and satisfies the condition 3. The Au content in the electrode of the main pump cell 21 in the example 2 is 0.5, and the Au content in the electrode of the measurement pump cell 41 is 0.0. In other words, in the example 2, the Au content in the electrode of the measurement pump cell 41 (the Au content in the measurement electrode 44) is lower than the Au content in the electrode of the main pump cell 21 (the Au content in the inner pump electrode 22), and satisfies the condition 5. The inter-electrode distance in the main pump cell 21 in the example 2 is 0.2, and the inter-electrode distance in the measurement pump cell 41 is 0.2. In other words, in the example 2, the inter-electrode distance in the measurement pump cell 41 (the distance between the measurement electrode 44 and the outer pump electrode 23) is equal to the inter-electrode distance in the main pump cell 21 (the distance between the inner pump electrode 22 and the outer pump electrode 23), and does not satisfy the condition 6. The ratio between precious metal and zirconia in the electrode of the main pump cell 21 in the example 2 is 85:15, and the ratio between precious metal and zirconia in the electrode of the measurement pump cell 41 is 85:15. In other words, in the example 2, the ratio of precious metal to zirconia in the electrode of the measurement pump cell 41 (the ratio of precious metal to zirconia in the measurement electrode 44) is equal to the ratio of precious metal to zirconia in the electrode of the main pump cell 21 (the ratio of precious metal to zirconia in the inner pump electrode 22), and does not satisfy the condition 4.

Accordingly, of the aforementioned conditions 1 to 6, the conditions 2, 4, and 6 are not satisfied but the conditions 1, 3, and 5 are satisfied in the example 2. Also, “RATIO OF SLOPE OF CELL RESISTANCE TO INPUT POWER TO HEATER (MAIN/MEASUREMENT)” in the example 2 is 30. In other words, in the gas sensor according to the example 2, the slope of the cell resistance of the main pump cell 21 (the slope of the cell resistance with respect to the input power to the heater 70) takes a value that is 1.5 to 1000 times the slope of the cell resistance of the measurement pump cell 41. The results of the offset change evaluations 1 and 2 for the gas sensor according to the example 2 were both A, i.e., the best evaluation result.

### Regarding Example 3

The electrode area in the main pump cell 21 in the example 3 is 5.0, and the electrode area in the measurement pump cell 41 is 5.0. In other words, in the example 3, the electrode area in the measurement pump cell 41 (the area of the measurement electrode 44) is equal to the electrode area in the main pump cell 21 (the area of the inner pump electrode 22), and does not satisfy the condition 1. The electrode thickness in the main pump cell 21 in the example 3 is 15, and the electrode thickness in the measurement pump cell 41 is 15. In other words, in the example 3, the electrode thickness in the measurement pump cell 41 (the thickness of the measurement electrode 44) is equal to the electrode thickness in the main pump cell 21 (the thickness of the inner pump electrode 22), and does not satisfy the condition 2. The electrode porosity in the main pump cell 21 in the example 3 is 15, and the electrode porosity in the measurement pump cell 41 is 15. In other words, in the example 3, the electrode porosity in the measurement pump cell 41 (the porosity of the measurement electrode 44) is equal to the electrode porosity in the main pump cell 21 (the porosity of the inner pump electrode 22), and does not satisfy the condition 3. The Au content in the electrode of the main pump cell 21 in the example 3 is 1.0, and the Au content in the electrode of the measurement pump cell 41 is 0.0. In other words, in the example 3, the Au content in the electrode of the measurement pump cell 41 (the Au content in the measurement electrode 44) is lower than the Au content in the electrode of the main pump cell 21 (the Au content in the inner pump electrode 22), and satisfies the condition 5. The inter-electrode distance in the main pump cell 21 in the example 3 is 0.2, and the inter-electrode distance in the measurement pump cell 41 is 0.2. In other words, in the example 3, the inter-electrode distance in the measurement pump cell 41 (the distance between the measurement electrode 44 and the outer pump electrode 23) is equal to the inter-electrode distance in the main pump cell 21 (the distance between the inner pump electrode 22 and the outer pump electrode 23), and does not satisfy the condition 6. The ratio between precious metal and zirconia in the electrode of the main pump cell 21 in the example 3 is 85:15, and the ratio between precious metal and zirconia in the electrode of the measurement pump cell 41 is 85:15. In other words, in the example 3, the ratio of precious metal to zirconia in the electrode of the measurement pump cell 41 (the ratio of precious metal to zirconia in the measurement electrode 44) is equal to the ratio of precious metal to zirconia in the electrode of the main pump cell 21 (the ratio of precious metal to zirconia in the inner pump electrode 22), and does not satisfy the condition 4.

Accordingly, of the aforementioned conditions 1 to 6, the conditions 1, 2, 3, 4, and 6 are not satisfied but the condition 5 is satisfied in the example 3. Also, “RATIO OF SLOPE OF CELL RESISTANCE TO INPUT POWER TO HEATER (MAIN/MEASUREMENT)” in the example 3 is 2. In other words, in the gas sensor according to the example 3, the slope of the cell resistance (the slope of the cell resistance with respect to the input power to the heater 70) of the main pump cell 21 takes a value that is 1.5 to 1000 times the slope of the cell resistance of the measurement pump cell 41. The results of the offset change evaluations 1 and 2 for the gas sensor according to the example 3 were both A, i.e., the best evaluation result.

### Regarding Example 4

The electrode area in the main pump cell 21 in the example 4 is 5.0, and the electrode area in the measurement pump cell 41 is 12.0. In other words, in the example 4, the electrode area in the measurement pump cell 41 (the area of the measurement electrode 44) is larger than the electrode area in the main pump cell 21 (the area of the inner pump electrode 22), and satisfies the condition 1. The electrode thickness in the main pump cell 21 in the example 4 is 15, and the electrode thickness in the measurement pump cell 41 is 15. In other words, in the example 4, the electrode thickness in the measurement pump cell 41 (the thickness of the measurement electrode 44) is equal to the electrode thickness in the main pump cell 21 (the thickness of the inner pump electrode 22), and does not satisfy the condition 2. The electrode porosity in the main pump cell 21 in the example 4 is 20, and the electrode porosity in the measurement pump cell 41 is 20. In other words, in the example 4, the electrode porosity in the measurement pump cell 41 (the porosity of the measurement electrode 44) is equal to the electrode porosity in the main pump cell 21 (the porosity of the inner pump electrode 22), and does not satisfy the condition 3. The Au content in the electrode of the main pump cell 21 in the example 4 is 0.0, and the Au content in the electrode of the measurement pump cell 41 is 0.0. In other words, in the example 4, the Au content in the electrode of the measurement pump cell 41 (the Au content in the measurement electrode 44) is equal to the Au content in the electrode of the main pump cell 21 (the Au content in the inner pump electrode 22), and does not satisfy the condition 5. The inter-electrode distance in the main pump cell 21 in the example 4 is 0.2, and the inter-electrode distance in the measurement pump cell 41 is 0.2. In other words, in the example 4, the inter-electrode distance in the measurement pump cell 41 (the distance between the measurement electrode 44 and the outer pump electrode 23) is equal to the inter-electrode distance in the main pump cell 21 (the distance between the inner pump electrode 22 and the outer pump electrode 23), and does not satisfy the condition 6. The ratio between precious metal and zirconia in the electrode of the main pump cell 21 in the example 4 is 85:15, and the ratio between precious metal and zirconia in the electrode of the measurement pump cell 41 is 85:15. In other words, in the example 4, the ratio of precious metal to zirconia in the electrode of the measurement pump cell 41 (the ratio of precious metal to zirconia in the measurement electrode 44) is equal to the ratio of precious metal to zirconia in the electrode of the main pump cell 21 (the ratio of precious metal to zirconia in the inner pump electrode 22), and does not satisfy the condition 4.

Accordingly, of the aforementioned conditions 1 to 6, the conditions 2, 3, 4, 5, and 6 are not satisfied but the condition 1 is satisfied in the example 4. Also, “RATIO OF SLOPE OF CELL RESISTANCE TO INPUT POWER TO HEATER (MAIN/MEASUREMENT)” in the example 4 is 2. In other words, in the gas sensor according to the example 4, the slope of the cell resistance of the main pump cell 21 (the slope of the cell resistance with respect to the input power to the heater 70) takes a value that is 1.5 to 1000 times the slope of the cell resistance of the measurement pump cell 41. The results of the offset change evaluations 1 and 2 for the gas sensor according to the example 4 were both A, i.e., the best evaluation result.

### Regarding Example 5

The electrode area in the main pump cell 21 in the example 5 is 10.0, and the electrode area in the measurement pump cell 41 is 10.0. In other words, in the example 5, the electrode area in the measurement pump cell 41 (the area of the measurement electrode 44) is equal to the electrode area in the main pump cell 21 (the area of the inner pump electrode 22), and does not satisfy the condition 1. The electrode thickness in the main pump cell 21 in the example 5 is 10, and the electrode thickness in the measurement pump cell 41 is 25. In other words, in the example 5, the electrode of the measurement pump cell 41 (the measurement electrode 44) is thicker than the electrode of the main pump cell 21 (the inner pump electrode 22), and satisfies the condition 2. The electrode porosity in the main pump cell 21 in the example 5 is 20, and the electrode porosity in the measurement pump cell 41 is 20. In other words, in the example 5, the electrode porosity in the measurement pump cell 41 (the porosity of the measurement electrode 44) is equal to the electrode porosity in the main pump cell 21 (the porosity of the inner pump electrode 22), and does not satisfy the condition 3. The Au content in the electrode of the main pump cell 21 in the example 5 is 0.0, and the Au content in the electrode of the measurement pump cell 41 is 0.0. In other words, in the example 5, the Au content in the electrode of the measurement pump cell 41 (the Au content in the measurement electrode 44) is equal to the Au content in the electrode of the main pump cell 21 (the Au content in the inner pump electrode 22), and does not satisfy the condition 5. The inter-electrode distance in the main pump cell 21 in the example 5 is 0.2, and the inter-electrode distance in the measurement pump cell 41 is 0.2. In other words, in the example 5, the inter-electrode distance in the measurement pump cell 41 (the distance between the measurement electrode 44 and the outer pump electrode 23) is equal to the inter-electrode distance in the main pump cell 21 (the distance between the inner pump electrode 22 and the outer pump electrode 23), and does not satisfy the condition 6. The ratio between precious metal and zirconia in the electrode of the main pump cell 21 in the example 5 is 85:15, and the ratio between precious metal and zirconia in the electrode of the measurement pump cell 41 is 85:15. In other words, in the example 5, the ratio of precious metal to zirconia in the electrode of the measurement pump cell 41 (the ratio of precious metal to zirconia in the measurement electrode 44) is equal to the ratio of precious metal to zirconia in the electrode of the main pump cell 21 (the ratio of precious metal to zirconia in the inner pump electrode 22), and does not satisfy the condition 4.

Accordingly, of the aforementioned conditions 1 to 6, the conditions 1, 3, 4, 5, and 6 are not satisfied but the condition 2 is satisfied in the example 5. Also, “RATIO OF SLOPE OF CELL RESISTANCE TO INPUT POWER TO HEATER (MAIN/MEASUREMENT)” in the example 5 is 1.5. In other words, in the gas sensor according to the example 5, the slope of the cell resistance of the main pump cell 21 (the slope of the cell resistance with respect to the input power to the heater 70) takes a value that is 1.5 to 1000 times the slope of the cell resistance of the measurement pump cell 41. The results of the offset change evaluations 1 and 2 for the gas sensor according to the example 5 were both A, i.e., the best evaluation result.

### Regarding Example 6

The electrode area in the main pump cell 21 in the example 6 is 10.0, and the electrode area in the measurement pump cell 41 is 10.0. In other words, in the example 6, the electrode area in the measurement pump cell 41 (the area of the measurement electrode 44) is equal to the electrode area in the main pump cell 21 (the area of the inner pump electrode 22), and does not satisfy the condition 1. The electrode thickness in the main pump cell 21 in the example 6 is 15, and the electrode thickness in the measurement pump cell 41 is 15. In other words, in the example 6, the electrode thickness in the measurement pump cell 41 (the thickness of the measurement electrode 44) is equal to the electrode thickness in the main pump cell 21 (the thickness of the inner pump electrode 22), and does not satisfy the condition 2. The electrode porosity in the main pump cell 21 in the example 6 is 15, and the electrode porosity in the measurement pump cell 41 is 15. In other words, in the example 6, the electrode porosity in the measurement pump cell 41 (the porosity of the measurement electrode 44) is equal to the electrode porosity in the main pump cell 21 (the porosity of the inner pump electrode 22), and does not satisfy the condition 3. The Au content in the electrode of the main pump cell 21 in the example 6 is 0.0, and the Au content in the electrode of the measurement pump cell 41 is 0.0. In other words, in the example 6, the Au content in the electrode of the measurement pump cell 41 (the Au content in the measurement electrode 44) is equal to the Au content in the electrode of the main pump cell 21 (the Au content in the inner pump electrode 22), and does not satisfy the condition 5. The inter-electrode distance in the main pump cell 21 in the example 6 is 0.4, and the inter-electrode distance in the measurement pump cell 41 is 0.2. In other words, in the example 6, the inter-electrode distance in the measurement pump cell 41 (the distance between the measurement electrode 44 and the outer pump electrode 23) is smaller than the inter-electrode distance in the main pump cell 21 (the distance between the inner pump electrode 22 and the outer pump electrode 23), and satisfies the condition 6. The ratio between precious metal and zirconia in the electrode of the main pump cell 21 in the example 6 is 85:15, and the ratio between precious metal and zirconia in the electrode of the measurement pump cell 41 is 85:15. In other words, in the example 6, the ratio of precious metal to zirconia in the electrode of the measurement pump cell 41 (the ratio of precious metal to zirconia in the measurement electrode 44) is equal to the ratio of precious metal to zirconia in the electrode of the main pump cell 21 (the ratio of precious metal to zirconia in the inner pump electrode 22), and does not satisfy the condition 4.

Accordingly, of the aforementioned conditions 1 to 6, the conditions 1, 2, 3, 4, and 5 are not satisfied but the condition 6 is satisfied in the example 6. Also, “RATIO OF SLOPE OF CELL RESISTANCE TO INPUT POWER TO HEATER (MAIN/MEASUREMENT)” in the example 6 is 1.8. In other words, in the gas sensor according to the example 6, the slope of the cell resistance (the slope of the cell resistance with respect to the input power to the heater 70) of the main pump cell 21 takes a value that is 1.5 to 1000 times the slope of the cell resistance of the measurement pump cell 41. The results of the offset change evaluations 1 and 2 for the gas sensor according to the example 6 were both A, i.e., the best evaluation result.

### Regarding Example 7

The electrode area in the main pump cell 21 in the example 7 is 10.0, and the electrode area in the measurement pump cell 41 is 10.0. In other words, in the example 7, the electrode area in the measurement pump cell 41 (the area of the measurement electrode 44) is equal to the electrode area in the main pump cell 21 (the area of the inner pump electrode 22), and does not satisfy the condition 1. The electrode thickness in the main pump cell 21 in the example 7 is 15, and the electrode thickness in the measurement pump cell 41 is 15. In other words, in the example 7, the electrode thickness in the measurement pump cell 41 (the thickness of the measurement electrode 44) is equal to the electrode thickness in the main pump cell 21 (the thickness of the inner pump electrode 22), and does not satisfy the condition 2. The electrode porosity in the main pump cell 21 in the example 7 is 15, and the electrode porosity in the measurement pump cell 41 is 15. In other words, in the example 7, the electrode porosity in the measurement pump cell 41 (the porosity of the measurement electrode 44) is equal to the electrode porosity in the main pump cell 21 (the porosity of the inner pump electrode 22), and does not satisfy the condition 3. The Au content in the electrode of the main pump cell 21 in the example 7 is 0.0, and the Au content in the electrode of the measurement pump cell 41 is 0.0. In other words, in the example 7, the Au content in the electrode of the measurement pump cell 41 (the Au content in the measurement electrode 44) is equal to the Au content in the electrode of the main pump cell 21 (the Au content in the inner pump electrode 22), and does not satisfy the condition 5. The inter-electrode distance in the main pump cell 21 in the example 7 is 0.2, and the inter-electrode distance in the measurement pump cell 41 is 0.2. In other words, in the example 7, the inter-electrode distance in the measurement pump cell 41 (the distance between the measurement electrode 44 and the outer pump electrode 23) is equal to the inter-electrode distance in the main pump cell 21 (the distance between the inner pump electrode 22 and the outer pump electrode 23), and does not satisfy the condition 6. The ratio between precious metal and zirconia in the electrode of the main pump cell 21 in the example 7 is 70:30, and the ratio between precious metal and zirconia in the electrode of the measurement pump cell 41 is 85:15. In other words, in the example 7, the ratio of precious metal to zirconia in the electrode of the measurement pump cell 41 (the ratio of precious metal to zirconia in the measurement electrode 44) is higher than the ratio of precious metal to zirconia in the electrode of the main pump cell 21 (the ratio of precious metal to zirconia in the inner pump electrode 22), and satisfies the condition 4.

Accordingly, of the aforementioned conditions 1 to 6, the conditions 1, 2, 3, 5, and 6 are not satisfied but the condition 4 is satisfied in the example 7. Also, “RATIO OF SLOPE OF CELL RESISTANCE TO INPUT POWER TO HEATER (MAIN/MEASUREMENT)” in the example 7 is 2.5. In other words, in the gas sensor according to the example 7, the slope of the cell resistance of the main pump cell 21 (the slope of the cell resistance with respect to the input power to the heater 70) takes a value that is 1.5 to 1000 times the slope of the cell resistance of the measurement pump cell 41. The results of the offset change evaluations 1 and 2 for the gas sensor according to the example 7 were both A, i.e., the best evaluation result.

### Regarding Example 8

The electrode area in the main pump cell 21 in the example 8 is 10.0, and the electrode area in the measurement pump cell 41 is 10.0. In other words, in the example 8, the electrode area in the measurement pump cell 41 (the area of the measurement electrode 44) is equal to the electrode area in the main pump cell 21 (the area of the inner pump electrode 22), and does not satisfy the condition 1. The electrode thickness in the main pump cell 21 in the example 8 is 15, and the electrode thickness in the measurement pump cell 41 is 15. In other words, in the example 8, the electrode thickness in the measurement pump cell 41 (the thickness of the measurement electrode 44) is equal to the electrode thickness in the main pump cell 21 (the thickness of the inner pump electrode 22), and does not satisfy the condition 2. The electrode porosity in the main pump cell 21 in the example 8 is 50, and the electrode porosity in the measurement pump cell 41 is 10. In other words, in the example 8, the electrode porosity in the measurement pump cell 41 (the porosity of the measurement electrode 44) is lower than the electrode porosity in the main pump cell 21 (the porosity of the inner pump electrode 22), and satisfies the condition 3. The Au content in the electrode of the main pump cell 21 in the example 8 is 0.0, and the Au content in the electrode of the measurement pump cell 41 is 0.0. In other words, in the example 8, the Au content in the electrode of the measurement pump cell 41 (the Au content in the measurement electrode 44) is equal to the Au content in the electrode of the main pump cell 21 (the Au content in the inner pump electrode 22), and does not satisfy the condition 5. The inter-electrode distance in the main pump cell 21 in the example 8 is 0.2, and the inter-electrode distance in the measurement pump cell 41 is 0.2. In other words, in the example 8, the inter-electrode distance in the measurement pump cell 41 (the distance between the measurement electrode 44 and the outer pump electrode 23) is equal to the inter-electrode distance in the main pump cell 21 (the distance between the inner pump electrode 22 and the outer pump electrode 23), and does not satisfy the condition 6. The ratio between precious metal and zirconia in the electrode of the main pump cell 21 in the example 8 is 85:15, and the ratio between precious metal and zirconia in the electrode of the measurement pump cell 41 is 85:15. In other words, in the example 8, the ratio of precious metal to zirconia in the electrode of the measurement pump cell 41 (the ratio of precious metal to zirconia in the measurement electrode 44) is equal to the ratio of precious metal to zirconia in the electrode of the main pump cell 21 (the ratio of precious metal to zirconia in the inner pump electrode 22), and does not satisfy the condition 4.

Accordingly, of the aforementioned conditions 1 to 6, the conditions 1, 2, 4, 5, and 6 are not satisfied but the condition 3 is satisfied in the example 8. Also, “RATIO OF SLOPE OF CELL RESISTANCE TO INPUT POWER TO HEATER (MAIN/MEASUREMENT)” in the example 8 is 1.8. In other words, in the gas sensor according to the example 8, the slope of the cell resistance of the main pump cell 21 (the slope of the cell resistance with respect to the input power to the heater 70) takes a value that is 1.5 to 1000 times the slope of the cell resistance of the measurement pump cell 41. The results of the offset change evaluations 1 and 2 for the gas sensor according to the example 8 were both A, i.e., the best evaluation result.

### Regarding Example 9

The electrode area in the main pump cell 21 in the example 9 is 7.5, and the electrode area in the measurement pump cell 41 is 10.0. In other words, in the example 9, the electrode area in the measurement pump cell 41 (the area of the measurement electrode 44) is larger than the electrode area in the main pump cell 21 (the area of the inner pump electrode 22), and satisfies the condition 1. The electrode thickness in the main pump cell 21 in the example 9 is 15, and the electrode thickness in the measurement pump cell 41 is 40. In other words, in the example 9, the electrode of the measurement pump cell 41 (the measurement electrode 44) is thicker than the electrode of the main pump cell 21 (the inner pump electrode 22), and satisfies the condition 2. The electrode porosity in the main pump cell 21 in the example 9 is 15, and the electrode porosity in the measurement pump cell 41 is 5. In other words, in the example 9, the electrode porosity in the measurement pump cell 41 (the porosity of the measurement electrode 44) is lower than the electrode porosity in the main pump cell 21 (the porosity of the inner pump electrode 22), and satisfies the condition 3. The Au content in the electrode of the main pump cell 21 in the example 9 is 1.0, and the Au content in the electrode of the measurement pump cell 41 is 0.0. In other words, in the example 9, the Au content in the electrode of the measurement pump cell 41 (the Au content in the measurement electrode 44) is lower than the Au content in the electrode of the main pump cell 21 (the Au content in the inner pump electrode 22), and satisfies the condition 5. The inter-electrode distance in the main pump cell 21 in the example 9 is 0.4, and the inter-electrode distance in the measurement pump cell 41 is 0.2. In other words, in the example 9, the inter-electrode distance in the measurement pump cell 41 (the distance between the measurement electrode 44 and the outer pump electrode 23) is smaller than the inter-electrode distance in the main pump cell 21 (the distance between the inner pump electrode 22 and the outer pump electrode 23), and satisfies the condition 6. The ratio between precious metal and zirconia in the electrode of the main pump cell 21 in the example 9 is 85:15, and the ratio between precious metal and zirconia in the electrode of the measurement pump cell 41 is 85:15. In other words, in the example 9, the ratio of precious metal to zirconia in the electrode of the measurement pump cell 41 (the ratio of precious metal to zirconia in the measurement electrode 44) is equal to the ratio of precious metal to zirconia in the electrode of the main pump cell 21 (the ratio of precious metal to zirconia in the inner pump electrode 22), and does not satisfy the condition 4.

Accordingly, of the aforementioned conditions 1 to 6, the condition 4 is not satisfied but the conditions 1, 2, 3, 5, and 6 are satisfied in the example 9. Also, “RATIO OF SLOPE OF CELL RESISTANCE TO INPUT POWER TO HEATER (MAIN/MEASUREMENT)” in the example 9 is 10. In other words, in the gas sensor according to the example 9, the slope of the cell resistance (the slope of the cell resistance with respect to the input power to the heater 70) of the main pump cell 21 takes a value that is 1.5 to 1000 times the slope of the cell resistance of the measurement pump cell 41. The results of the offset change evaluations 1 and 2 for the gas sensor according to the example 9 were both A, i.e., the best evaluation result.

### Regarding Example 10

The electrode area in the main pump cell 21 (“MAIN” below “AREA”) in the example 10 is 1.5, and the electrode area in the measurement pump cell 41 (“MEASUREMENT” below “AREA”) is 7.5. In other words, in the example 10, the electrode area in the measurement pump cell 41 (the area of the measurement electrode 44) is larger than the electrode area in the main pump cell 21 (the area of the inner pump electrode 22), and satisfies the condition 1. The electrode thickness in the main pump cell 21 (“MAIN” below “THICKNESS”) in the example 10 is 15, and the electrode thickness in the measurement pump cell 41 (“MEASUREMENT” below “THICKNESS”) is 25. In other words, in the example 10, the electrode of the measurement pump cell 41 (the measurement electrode 44) is thicker than the electrode of the main pump cell 21 (the inner pump electrode 22), and satisfies the condition 2. The electrode porosity in the main pump cell 21 (“MAIN” below “POROSITY”) in the example 10 is 10, and the electrode porosity in the measurement pump cell 41 (“MEASUREMENT” below “POROSITY”) is 5. In other words, in the example 10, the electrode porosity in the measurement pump cell 41 (the porosity of the measurement electrode 44) is lower than the electrode porosity in the main pump cell 21 (the porosity of the inner pump electrode 22), and satisfies the condition 3. The Au content in the electrode of the main pump cell 21 (“MAIN” below “Au CONTENT”) in the example 10 is 1.0, and the Au content in the electrode of the measurement pump cell 41 (“MEASUREMENT” below “Au CONTENT”) is 0.0. In other words, in the example 10, the Au content in the electrode of the measurement pump cell 41 (the Au content in the measurement electrode 44) is lower than the Au content in the electrode of the main pump cell 21 (the Au content in the inner pump electrode 22), and satisfies the condition 5. The inter-electrode distance in the main pump cell 21 (“MAIN” below “INTER-ELECTRODE DISTANCE”) in the example 10 is 0.4, and the inter-electrode distance in the measurement pump cell 41 (“MEASUREMENT” below “INTER-ELECTRODE DISTANCE”) is 0.2. In other words, in the example 10, the inter-electrode distance in the measurement pump cell 41 (the distance between the measurement electrode 44 and the outer pump electrode 23) is smaller than the inter-electrode distance in the main pump cell 21 (the distance between the inner pump electrode 22 and the outer pump electrode 23), and satisfies the condition 6. The ratio between precious metal and zirconia in the electrode of the main pump cell 21 (“MAIN” below “PRECIOUS METAL/ZIRCONIA RATIO”) in the example 10 is 70:30, and the ratio between precious metal and zirconia in the electrode of the measurement pump cell 41 (“MEASUREMENT” below “PRECIOUS METAL/ZIRCONIA RATIO”) is 85:15. In other words, in the example 10, the ratio of precious metal to zirconia in the electrode of the measurement pump cell 41 (the ratio of precious metal to zirconia in the measurement electrode 44) is higher than the ratio of precious metal to zirconia in the electrode of the main pump cell 21 (the ratio of precious metal to zirconia in the inner pump electrode 22), and satisfies the condition 4.

Accordingly, all of the aforementioned conditions 1 to 6 are satisfied in the example 10. Also, “RATIO OF SLOPE OF CELL RESISTANCE TO INPUT POWER TO HEATER (MAIN/MEASUREMENT)” in the example 10 is 1000. In other words, in the gas sensor according to the example 10, the slope of the cell resistance of the main pump cell 21 (the slope of the cell resistance with respect to the input power to the heater 70) takes a value that is 1.5 to 1000 times the slope of the cell resistance of the measurement pump cell 41. The results of the offset change evaluations 1 and 2 for the gas sensor according to the example 10 were both A, i.e., the best evaluation result.

### Regarding Example 11

The electrode area in the main pump cell 21 (“MAIN” below “AREA”) in the example 11 is 6.0, and the electrode area in the measurement pump cell 41 (“MEASUREMENT” below “AREA”) is 5.0. In other words, in the example 11, the electrode area in the measurement pump cell 41 (the area of the measurement electrode 44) is smaller than the electrode area in the main pump cell 21 (the area of the inner pump electrode 22), and does not satisfy the condition 1. The electrode thickness in the main pump cell 21 (“MAIN” below “THICKNESS”) in the example 11 is 10, and the electrode thickness in the measurement pump cell 41 (“MEASUREMENT” below “THICKNESS”) is 20. In other words, in the example 11, the electrode of the measurement pump cell 41 (the measurement electrode 44) is thicker than the electrode of the main pump cell 21 (the inner pump electrode 22), and satisfies the condition 2. The electrode porosity in the main pump cell 21 (“MAIN” below “POROSITY”) in the example 11 is 20, and the electrode porosity in the measurement pump cell 41 (“MEASUREMENT” below “POROSITY”) is 20. In other words, in the example 11, the electrode porosity in the measurement pump cell 41 (the porosity of the measurement electrode 44) is equal to the electrode porosity in the main pump cell 21 (the porosity of the inner pump electrode 22), and does not satisfy the condition 3. The Au content in the electrode of the main pump cell 21 (“MAIN” below “Au CONTENT”) in the example 11 is 0.0, and the Au content in the electrode of the measurement pump cell 41 (“MEASUREMENT” below “Au CONTENT”) is 0.0. In other words, in the example 11, the Au content in the electrode of the measurement pump cell 41 (the Au content in the measurement electrode 44) is equal to the Au content in the electrode of the main pump cell 21 (the Au content in the inner pump electrode 22), and does not satisfy the condition 5. The inter-electrode distance in the main pump cell 21 (“MAIN” below “INTER-ELECTRODE DISTANCE”) in the example 11 is 0.2, and the inter-electrode distance in the measurement pump cell 41 (“MEASUREMENT” below “INTER-ELECTRODE DISTANCE”) is 0.2. In other words, in the example 11, the inter-electrode distance in the measurement pump cell 41 (the distance between the measurement electrode 44 and the outer pump electrode 23) is equal to the inter-electrode distance in the main pump cell 21 (the distance between the inner pump electrode 22 and the outer pump electrode 23), and does not satisfy the condition 6. The ratio between precious metal and zirconia in the electrode of the main pump cell 21 (“MAIN” below “PRECIOUS METAL/ZIRCONIA RATIO”) in the example 11 is 85:15, and the ratio between precious metal and zirconia in the electrode of the measurement pump cell 41 (“MEASUREMENT” below “PRECIOUS METAL/ZIRCONIA RATIO”) is 85:15. In other words, in the example 11, the ratio of precious metal to zirconia in the electrode of the measurement pump cell 41 (the ratio of precious metal to zirconia in the measurement electrode 44) is equal to the ratio of precious metal to zirconia in the electrode of the main pump cell 21 (the ratio of precious metal to zirconia in the inner pump electrode 22), and does not satisfy the condition 4.

Accordingly, of the aforementioned conditions 1 to 6, the condition 2 is satisfied but the conditions 1, 3, 4, 5, and 6 are not satisfied in the example 11. Also, “RATIO OF SLOPE OF CELL RESISTANCE TO INPUT POWER TO HEATER (MAIN/MEASUREMENT)” in the example 11 is 1.2. In other words, in the gas sensor according to the example 11, the slope of the cell resistance (the slope of the cell resistance with respect to the input power to the heater 70) of the main pump cell 21 does not take a value that is 1.5 to 1000 times the slope of the cell resistance of the measurement pump cell 41. However, in the example 11, the slope of the cell resistance of the adjustment pump cell (the main pump cell 21) is larger than the slope of the cell resistance of the measurement pump cell 41. Despite the results of the offset change evaluations 1 and 2 for the gas sensor according to the example 11 not being the best evaluation result A, they were both B.

### Regarding Example 12

The electrode area in the main pump cell 21 (“MAIN” below “AREA”) in the example 12 is 1.0, and the electrode area in the measurement pump cell 41 (“MEASUREMENT” below “AREA”) is 10.0. In other words, in the example 12, the electrode area in the measurement pump cell 41 (the area of the measurement electrode 44) is larger than the electrode area in the main pump cell 21 (the area of the inner pump electrode 22), and satisfies the condition 1. The electrode thickness in the main pump cell 21 (“MAIN” below “THICKNESS”) in the example 12 is 15, and the electrode thickness in the measurement pump cell 41 (“MEASUREMENT” below “THICKNESS”) is 25. In other words, in the example 12, the electrode of the measurement pump cell 41 (the measurement electrode 44) is thicker than the electrode of the main pump cell 21 (the inner pump electrode 22), and satisfies the condition 2. The electrode porosity in the main pump cell 21 (“MAIN” below “POROSITY”) in the example 12 is 10, and the electrode porosity in the measurement pump cell 41 (“MEASUREMENT” below “POROSITY”) is 5. In other words, in the example 12, the electrode porosity in the measurement pump cell 41 (the porosity of the measurement electrode 44) is lower than the electrode porosity in the main pump cell 21 (the porosity of the inner pump electrode 22), and satisfies the condition 3. The Au content in the electrode of the main pump cell 21 (“MAIN” below “Au CONTENT”) in the example 12 is 1.0, and the Au content in the electrode of the measurement pump cell 41 (“MEASUREMENT” below “Au CONTENT”) is 0.0. In other words, in the example 12, the Au content in the electrode of the measurement pump cell 41 (the Au content in the measurement electrode 44) is lower than the Au content in the electrode of the main pump cell 21 (the Au content in the inner pump electrode 22), and satisfies the condition 5. The inter-electrode distance in the main pump cell 21 (“MAIN” below “INTER-ELECTRODE DISTANCE”) in the example 12 is 0.4, and the inter-electrode distance in the measurement pump cell 41 (“MEASUREMENT” below “INTER-ELECTRODE DISTANCE”) is 0.2. In other words, in the example 12, the inter-electrode distance in the measurement pump cell 41 (the distance between the measurement electrode 44 and the outer pump electrode 23) is smaller than the inter-electrode distance in the main pump cell 21 (the distance between the inner pump electrode 22 and the outer pump electrode 23), and satisfies the condition 6. The ratio between precious metal and zirconia in the electrode of the main pump cell 21 (“MAIN” below “PRECIOUS METAL/ZIRCONIA RATIO”) in the example 12 is 70:30, and the ratio between precious metal and zirconia in the electrode of the measurement pump cell 41 (“MEASUREMENT” below “PRECIOUS METAL/ZIRCONIA RATIO”) is 85:15. In other words, in the example 12, the ratio of precious metal to zirconia in the electrode of the measurement pump cell 41 (the ratio of precious metal to zirconia in the measurement electrode 44) is higher than the ratio of precious metal to zirconia in the electrode of the main pump cell 21 (the ratio of precious metal to zirconia in the inner pump electrode 22), and satisfies the condition 4.

Accordingly, all of the aforementioned conditions 1 to 6 are satisfied in the example 12. However, “RATIO OF SLOPE OF CELL RESISTANCE TO INPUT POWER TO HEATER (MAIN/MEASUREMENT)” in the example 12 is 2000. In other words, in the gas sensor according to the example 12, the slope of the cell resistance (the slope of the cell resistance with respect to the input power to the heater 70) of the main pump cell 21 does not take a value that is 1.5 to 1000 times the slope of the cell resistance of the measurement pump cell 41. However, in the example 12, the slope of the cell resistance of the adjustment pump cell (the main pump cell 21) is larger than the slope of the cell resistance of the measurement pump cell 41. The results of the offset change evaluations 1 and 2 for the gas sensor according to the example 12 were both B, although lower than the best evaluation result A.

### Regarding Comparative Example 1

The electrode area in the main pump cell 21 (“MAIN” below “AREA”) in the comparative example 1 is 12.0, and the electrode area in the measurement pump cell 41 (“MEASUREMENT” below “AREA”) is 1.0. In other words, in the comparative example 1, the electrode area in the measurement pump cell 41 (the area of the measurement electrode 44) is smaller than the electrode area in the main pump cell 21 (the area of the inner pump electrode 22), and does not satisfy the condition 1. The electrode thickness in the main pump cell 21 (“MAIN” below “THICKNESS”) in the comparative example 1 is 15, and the electrode thickness in the measurement pump cell 41 (“MEASUREMENT” below “THICKNESS”) is 15. In other words, in the comparative example 1, the electrode thickness in the measurement pump cell 41 (the thickness of the measurement electrode 44) is equal to the electrode thickness in the main pump cell 21 (the thickness of the inner pump electrode 22), and does not satisfy the condition 2. The electrode porosity in the main pump cell 21 (“MAIN” below “POROSITY”) in the comparative example 1 is 10, and the electrode porosity in the measurement pump cell 41 (“MEASUREMENT” below “POROSITY”) is 20. In other words, in the comparative example 1, the electrode porosity in the measurement pump cell 41 (the porosity of the measurement electrode 44) is higher than the electrode porosity in the main pump cell 21 (the porosity of the inner pump electrode 22), and does not satisfy the condition 3. The Au content in the electrode of the main pump cell 21 (“MAIN” below “Au CONTENT”) in the comparative example 1 is 0.5, and the Au content in the electrode of the measurement pump cell 41 (“MEASUREMENT” below “Au CONTENT”) is 0.0. In other words, in the comparative example 1, the Au content in the electrode of the measurement pump cell 41 (the Au content in the measurement electrode 44) is lower than the Au content in the electrode of the main pump cell 21 (the Au content in the inner pump electrode 22), and satisfies the condition 5. The inter-electrode distance in the main pump cell 21 in the comparative example 1 (“MAIN” below “INTER-ELECTRODE DISTANCE”) is 0.2, and the inter-electrode distance in the measurement pump cell 41 (“MEASUREMENT” below “INTER-ELECTRODE DISTANCE”) is 0.2. In other words, in the comparative example 1, the inter-electrode distance in the measurement pump cell 41 (the distance between the measurement electrode 44 and the outer pump electrode 23) is equal to the inter-electrode distance in the main pump cell 21 (the distance between the inner pump electrode 22 and the outer pump electrode 23), and does not satisfy the condition 6. The ratio between precious metal and zirconia in the electrode of the main pump cell 21 in the comparative example 1 (“MAIN” below “PRECIOUS METAL/ZIRCONIA RATIO”) is 85:15, and the ratio between precious metal and zirconia in the electrode of the measurement pump cell 41 (“MEASUREMENT” below “precious metal/zirconia”) is 85:15. In other words, in the comparative example 1, the ratio of precious metal to zirconia in the electrode of the measurement pump cell 41 (the ratio of precious metal to zirconia in the measurement electrode 44) is equal to the ratio of precious metal to zirconia in the electrode of the main pump cell 21 (the ratio of precious metal to zirconia in the inner pump electrode 22), and does not satisfy the condition 4.

Accordingly, of the aforementioned conditions 1 to 6, the condition 5 is satisfied but the conditions 1, 2, 3, 4, and 6 are not satisfied in the comparative example 1. Also, “RATIO OF SLOPE OF CELL RESISTANCE TO INPUT POWER TO HEATER (MAIN/MEASUREMENT)” in the comparative example 1 is 0.002. In other words, in the gas sensor in the comparative example 1, the slope of the cell resistance (the slope of the cell resistance with respect to the input power to the heater 70) of the main pump cell 21 does not take a value that is 1.5 to 1000 times the slope of the cell resistance of the measurement pump cell 41. Also, in the comparative example 1, the slope of the cell resistance of the adjustment pump cell (the main pump cell 21) is smaller than the slope of the cell resistance of the measurement pump cell 41. The results of the offset change evaluations 1 and 2 for the gas sensor according to the comparative example 1 were both F, i.e., the worst evaluation result.

### Regarding Comparative Example 2

The electrode area in the main pump cell 21 (“MAIN” below “AREA”) in the comparative example 2 is 5.0, and the electrode area in the measurement pump cell 41 (“MEASUREMENT” below “AREA”) is 5.0. In other words, in the comparative example 2, the electrode area in the measurement pump cell 41 (the area of the measurement electrode 44) is equal to the electrode area in the main pump cell 21 (the area of the inner pump electrode 22), and does not satisfy the condition 1. The electrode thickness in the main pump cell 21 (“MAIN” below “THICKNESS”) in the comparative example 2 is 15, and the electrode thickness in the measurement pump cell 41 (“MEASUREMENT” below “THICKNESS”) is 15. In other words, in the comparative example 2, the electrode thickness in the measurement pump cell 41 (the thickness of the measurement electrode 44) is equal to the electrode thickness in the main pump cell 21 (the thickness of the inner pump electrode 22), and does not satisfy the condition 2. The electrode porosity in the main pump cell 21 (“MAIN” below “POROSITY”) in the comparative example 2 is 15, and the electrode porosity in the measurement pump cell 41 (“MEASUREMENT” below “POROSITY”) is 15. In other words, in the comparative example 2, the electrode porosity in the measurement pump cell 41 (the porosity of the measurement electrode 44) is equal to the electrode porosity in the main pump cell 21 (the porosity of the inner pump electrode 22), and does not satisfy the condition 3. The Au content in the electrode of the main pump cell 21 (“MAIN” below “Au CONTENT”) in the comparative example 2 is 0.0, and the Au content in the electrode of the measurement pump cell 41 (“MEASUREMENT” below “Au CONTENT”) is 0.0. In other words, in the comparative example 2, the Au content in the electrode of the measurement pump cell 41 (the Au content in the measurement electrode 44) is equal to the Au content in the electrode of the main pump cell 21 (the Au content in the inner pump electrode 22), and does not satisfy the condition 5. The inter-electrode distance in the main pump cell 21 in the comparative example 2 (“MAIN” below “INTER-ELECTRODE DISTANCE”) is 0.2, and the inter-electrode distance in the measurement pump cell 41 (“MEASUREMENT” below “INTER-ELECTRODE DISTANCE”) is 0.2. In other words, in the comparative example 2, the inter-electrode distance in the measurement pump cell 41 (the distance between the measurement electrode 44 and the outer pump electrode 23) is equal to the inter-electrode distance in the main pump cell 21 (the distance between the inner pump electrode 22 and the outer pump electrode 23), and does not satisfy the condition 6. The ratio between precious metal and zirconia in the electrode of the main pump cell 21 in the comparative example 2 (“MAIN” below “PRECIOUS METAL/ZIRCONIA RATIO”) is 85:15, and the ratio between precious metal and zirconia in the electrode of the measurement pump cell 41 (“MEASUREMENT” below “PRECIOUS METAL/ZIRCONIA”) is 85:15. In other words, in the comparative example 2, the ratio of precious metal to zirconia in the electrode of the measurement pump cell 41 (the ratio of precious metal to zirconia in the measurement electrode 44) is equal to the ratio of precious metal to zirconia in the electrode of the main pump cell 21 (the ratio of precious metal to zirconia in the inner pump electrode 22), and does not satisfy the condition 4.

Accordingly, none of the aforementioned conditions 1 to 6 are satisfied in the comparative example 2. Also, “RATIO OF SLOPE OF CELL RESISTANCE TO INPUT POWER TO HEATER (MAIN/MEASUREMENT)” in the comparative example 2 is 1.0. In other words, in the gas sensor in the comparative example 2, the slope of the cell resistance (the slope of the cell resistance with respect to the input power to the heater 70) of the main pump cell 21 does not take a value that is 1.5 to 1000 times the slope of the cell resistance of the measurement pump cell 41. Also, in the comparative example 2, the slope of the cell resistance of the adjustment pump cell (the main pump cell 21) is equal to the slope of the cell resistance of the measurement pump cell 41. The results of the offset change evaluations 1 and 2 for the gas sensor according to the comparative example 2 were both F, i.e., the worst evaluation result.

As is understood from the comparison between the examples 1 to 12 and the comparative examples 1 and 2, the change in the offset value can be suppressed by making the slope of the cell resistance of the adjustment pump cell (the main pump cell 21 in the example in FIG. 6) larger than the slope of the cell resistance of the measurement pump cell 41. In other words, by making the slope of the cell resistance of the main pump cell 21 larger than the slope of the cell resistance of the measurement pump cell 41, highly accurate concentration measurement can be realized in both environments where the NOₓ concentration in the measurement target gas is high and environments where the concentration is low. Accordingly, the gas sensor according to this embodiment can realize highly accurate concentration measurement under both high and low concentrations. Also, as is understood from the comparison between the examples 1 to 10 and the examples 11 and 12, the change in the offset value can be effectively suppressed by setting the slope of the cell resistance of the adjustment pump cell to a value that is 1.5 to 1000 times the slope of the cell resistance of the measurement pump cell 41. Also, to make the slope of the cell resistance of the adjustment pump cell larger than the slope of the cell resistance of the measurement pump cell 41, it is effective to configure the adjustment pump cell and the measurement pump cell 41 so as to satisfy at least one of the conditions 1 to 6, as is understood from the examples 1 to 12 and the comparative example 2.

### Notes on Adjustment Pump Cell

Note that the inventor has also confirmed that, to make the slope of the cell resistance of the auxiliary pump cell 50 serving as the adjustment pump cell larger than the slope of the cell resistance of the measurement pump cell 41, it is effective to configure the auxiliary pump cell 50 and the measurement pump cell 41 so as to satisfy at least one of the conditions 1 to 6. In the case of using the auxiliary pump cell 50 as the adjustment pump cell, the condition 1 means the condition that the area of the measurement electrode 44 is larger than the area of the auxiliary pump electrode 51. The condition 2 means the condition that the measurement electrode 44 is thicker than the auxiliary pump electrode 51. The condition 3 means the condition that the porosity of the measurement electrode 44 is lower than the porosity of the auxiliary pump electrode 51. The condition 4 means the condition that the ratio of precious metal to zirconia in the measurement electrode 44 is higher than the ratio of precious metal to zirconia in the auxiliary pump electrode 51. The condition 5 means the condition that the Au content in the measurement electrode 44 is lower than the Au content in the auxiliary pump electrode 51. The condition 6 means the condition that the distance between the measurement electrode 44 and the outer pump electrode 23 is smaller (shorter) than the distance between the auxiliary pump electrode 51 and the outer pump electrode 23 (or the third electrode).

### LIST OF REFERENCE NUMERALS

S, S1 Gas sensor
100 Sensor element
111 Detection unit
112 Temperature adjustment unit (adjustment unit)
1 First substrate layer (solid electrolyte layer)
2 Second substrate layer (solid electrolyte layer)
3 Third substrate layer (solid electrolyte layer)
4 First solid electrolyte layer (solid electrolyte layer)
5 Spacer layer (solid electrolyte layer)
6 Second solid electrolyte layer (solid electrolyte layer)
7 Measurement target gas flow section (internal space)
44 Measurement electrode
23 Outer pump electrode
41 Measurement pump cell
70 Heater (heater unit)
22 Inner pump electrode
51 Auxiliary pump electrode (inner pump electrode)
21 Main pump cell (adjustment pump cell)
50 Auxiliary pump cell (adjustment pump cell)

What is claimed is:

1. A gas sensor comprising:
a sensor element formed by stacking a plurality of solid electrolyte layers having oxygen ion conductivity, the sensor element including:
an internal cavity into which a measurement target gas is to be introduced;
a measurement pump cell being an electrochemical pump cell including: a measurement electrode located in the internal cavity; an outer pump electrode located in a region different from the internal cavity; and a solid electrolyte layer, of the plurality of solid electrolyte layers, that is present between the measurement electrode and the outer pump electrode;
an adjustment pump cell being an electrochemical pump cell including: an inner pump electrode facing the internal cavity; the outer pump electrode, or a third electrode in contact with a solid electrolyte layer, of the plurality of solid electrolyte layers, and exposed to an external space; and a solid electrolyte layer, of the plurality of solid electrolyte layers, that is present between the inner pump electrode and the outer pump electrode or the third electrode; and
a heater unit embedded in the sensor element and configured to heat the sensor element to a specific temperature;
a detection unit configured to detect a value of cell resistance of the adjustment pump cell; and
an adjustment unit configured to adjust the specific temperature such that the value of the cell resistance of the adjustment pump cell detected by the detection unit is a predetermined value,
wherein a slope of the cell resistance of the adjustment pump cell with respect to input power to the heater unit is larger than a slope of cell resistance of the measurement pump cell with respect to the input power to the heater unit.

2. The gas sensor according to claim 1,
wherein the slope of the cell resistance of the adjustment pump cell with respect to the input power to the heater unit is 1.5 to 1000 times the slope of the cell resistance of the measurement pump cell with respect to the input power to the heater unit.

3. The gas sensor according to claim 1,
wherein the measurement electrode has an area larger than an area of the inner pump electrode.

4. The gas sensor according to claim 1,
wherein the measurement electrode is thicker than the inner pump electrode.

5. The gas sensor according to claim 1,
wherein the measurement electrode has a porosity lower than a porosity of the inner pump electrode.

6. The gas sensor according to claim 1,
wherein the measurement electrode and the inner pump electrode are cermet electrodes made of zirconia and precious metal, and
a ratio of precious metal to zirconia in the measurement electrode is higher than a ratio of precious metal to zirconia in the inner pump electrode.

7. The gas sensor according to claim 1,
wherein the measurement electrode has an Au content lower than an Au content in the inner pump electrode.

8. The gas sensor according to claim 1,
wherein a distance between the measurement electrode and the outer pump electrode is smaller than a distance between the inner pump electrode and the outer pump electrode or the third electrode.

9. A method for controlling a gas sensor including a sensor element formed by stacking a plurality of solid electrolyte layers having oxygen ion conductivity,
the sensor element including:
an internal cavity into which a measurement target gas is to be introduced;
a measurement pump cell being an electrochemical pump cell including: a measurement electrode located in the internal cavity; an outer pump electrode located in a region different from the internal cavity; and a solid electrolyte layer, of the plurality of solid electrolyte layers, that is present between the measurement electrode and the outer pump electrode;
an adjustment pump cell being an electrochemical pump cell including: an inner pump electrode facing the internal cavity; the outer pump electrode, or a third electrode in contact with a solid electrolyte layer, of the plurality of solid electrolyte layers, and exposed to an external space; and a solid electrolyte layer, of the plurality of solid electrolyte layers, that is present between the inner pump electrode and the outer pump electrode or the third electrode; and
a heater unit embedded in the sensor element and configured to heat the sensor element to a specific temperature,
the method comprising:
a detection step of detecting a value of cell resistance of the adjustment pump cell; and
an adjustment step of adjusting the specific temperature such that the value of the cell resistance of the adjustment pump cell detected in the detection step is a predetermined value,
wherein a slope of the cell resistance of the adjustment pump cell with respect to input power to the heater unit is larger than a slope of cell resistance of the measurement pump cell with respect to the input power to the heater unit.
